(19) United States
(12) Patent Application Publication
(10) Pub. No.: US 2026/0337372 A1
(43) Pub. Date: Oct. 29, 2026

(54) ANTIMICROBIAL POLYMER TECHNOLOGY

(71) Applicant: Zonova Technologies Ltd, London (GB)

(72) Inventors: Georgia FLEET, London (GB); Peter BRAWN, London (GB); Manish TRIVEDI, London (GB)

(73) Assignee: Zonova Technologies Ltd, London (GB)

(21) Appl. No.: 19/641,355

(22) Filed: Apr. 7, 2026

Related U.S. Application Data
(60) Provisional application No. 63/784,705, filed on Apr. 7, 2025.

(51) Int. Cl. C08K 3/22; A61B 17/88

(52) U.S. Cl. CPC C08K 3/22; A61B 17/8805; C08K 2003/222; C08K 2003/2241; C08K 2003/2248; C08K 2003/2258; C08K 2003/2265; C08K 2003/2286; C08K 2003/2296; C08K 2201/011

(57) ABSTRACT

The disclosure provides antimicrobial polymer compositions comprising a plurality of metallic nanoparticles, one or more photosensitizers, and a polymer, wherein the components are combined under high temperature or pressure conditions to form a medical device exhibiting antimicrobial activity under both light and dark conditions without requiring a light-activation source. The disclosure also provides methods of preparing an antimicrobial polymer composition and methods of manufacturing an antimicrobial device with the antimicrobial polymer composition by extrusion, injection molding, pouring, or a combination thereof. The disclosure further provides compositions and methods of preparing antimicrobial products wherein the products are a textile product or a non-textile polymeric article.

### CROSS REFERENCE TO RELATED APPLICATIONS

This application also claims the benefit of and priority to U.S. Provisional Application No. 63/784,705, filed on Apr. 7, 2025, and U.S. Provisional Application No. 63/944,160, filed on Dec. 18, 2025, which are incorporated by reference in their entirety.

### FIELD

This invention relates generally to antimicrobial polymer compositions and uses therefore, wherein the antimicrobial compositions are used in objects susceptible to risks of infection that can be harmful to humans or animals, such as in a medical environment.

### BACKGROUND

Patients who receive medical devices or implants are susceptible to the risk of infection from biofilm-forming microbial pathogens. These infections can occur in the bloodstream, surgical site, skin and soft tissue, respiratory, and urinary tract. Bacteria are able to form a biofilm on implanted devices at these sites due to the solid-liquid air interface provided by the device. The bacteria can adhere to the surface of the device, leading to an increased risk of nosocomial infections. Examples of devices include urinary catheters, intravascular catheters, and orthopedic implants. Commonly, nosocomial infections are linked to antibiotic-resistant microorganisms, demonstrating the need for a strategy to reduce infection without antibiotics (Liao W, et al. J. Microbiol. Immunol. Infect. 55: 12931300 (2022)).

Reactive oxygen species (ROS) have demonstrated significant antimicrobial action against biofilm-producing pathogens. (Dryden M. Curr. Opin. Infect. Dis. 30:143-149 (2017)). ROS include radical and non-radical oxygen-containing atoms and molecules, which can be formed as byproducts of aerobic respiration or from ROS-producing compounds. ROS-producing compounds include nanoparticles and photosensitizers, which have been used in clinical environments to reduce infection.

Photosensitizers, such as toluidine blue O, act as light-activated antimicrobial agents. Although they may have no antimicrobial activity at low concentrations in the dark, when irradiated with light of a certain wavelength (such as 633 nm for toluidine blue O) they can kill a wide range of microbes. The killing of microbes is thought to be due to the production of singlet oxygen upon irradiation of the compound. There is considerable interest in enhancing the activity of existing photosensitizers.

U.S. Pat. No. 8,580,309B2 describes antimicrobial mixtures comprising charge-stabilized metallic nanoparticles and a photosensitizer, and their use as light-activated antimicrobials. The authors described metallic nanoparticle-ligand-photosensitizer conjugates for use as light-activated antimicrobials capable of killing or preventing the growth of microbes on surfaces in a medical environment. This antimicrobial technology utilizes nanoparticles to augment the natural antimicrobial properties of the photosensitizer dye, which has not previously exhibited its own antimicrobial activity. However, these photosensitizer dyes are activated by light and, therefore, are not active in the dark. Medical devices in living subjects often sit within the body in the dark and cannot be activated by light. Therefore, there is a need for antimicrobial compositions that can be activated within a subject's body without a light source.

Although coating medical devices with antimicrobial agents is widely practiced, it has been determined to have potential drawbacks. For example, it often requires the use of solvents and a process to apply the coating. Also, since the antimicrobial agent is only present in the coating, the antimicrobial properties are localized at the surface upon which the coating is applied. Furthermore, the coating can wear off, resulting in the loss of the desired antimicrobial properties of the device and the risk of infection returning. Accordingly, it is desirable to provide an improved antimicrobial article that overcomes at least some of the drawbacks of the prior art.

Presented herein are antimicrobial polymer compositions that are active in both light and dark conditions, eliminating the need for a light source. These compositions can be used to manufacture antimicrobial medical devices.

### SUMMARY

In accordance with the purpose(s) of this invention, as embodied and broadly described herein, this invention, in some aspects, relates to an antimicrobial polymer composition comprising: a) a plurality of metallic nanoparticles; b) one or more photosensitizers; and c) a polymer; wherein the metallic nanoparticles and one or more photosensitizers are mixed to form a metallic nanoparticle-photosensitizer mixture; wherein the metallic nanoparticle-photosensitizer mixture is combined with the polymer under high temperature or pressure conditions to form a medical device; and wherein the medical device exhibits antimicrobial activity under both light and dark conditions without requiring a light-activation source.

In some embodiments, the plurality of metallic nanoparticles are selected from a group consisting of zinc oxide, zinc, gold, silver, copper, cupric oxide, magnesium oxide, iron (II,III) oxide, graphene oxide, titanium dioxide, tungsten (VI) oxide nanoparticles, alloys of two or more of these metals, or a combination thereof. In some embodiments, neither the plurality of metallic nanoparticles nor the one or more photosensitizers leaches from the composition. In some embodiments, the photosensitizer comprises about 0.05% to about 5% by weight of the total polymer. In some embodiments, the metallic nanoparticles comprise about 1% to about 10% by weight of the total polymer. In some embodiments, the plurality of nanoparticles comprises nanoparticles of less than 100 nm in diameter.

In some embodiments, the one or more photosensitizers are selected from the group consisting of toluidine blue O, methylene blue, dimethylmethylene blue, zinc phthalocyanine, silicon phthalocyanine, aluminum phthalocyanine, Rose Bengal, porphyrin, haematoporphyrin, deuteroporphyrin, tin chlorin e6, poly-lysine derivatives of tin chlorin e6, m-tetrahydroxyphenyl chlorin, benzoporphyrin derivatives, tin etiopurpurin, bacteriochlorins, phenazines, acriflavine, proflavin, acridine orange, aminacrine, texaphyrins, cyanines including merocyanine 540, anthracyclins including adriamycin and epirubicin, pheophorbides, sapphyrins, fullerene, perylenequinonoid pigments including hypericin and hypocrellin, gilvocarcins, terthiophenes, benzophenanthridines, tris(2,2′-bipyridine)ruthenium(II) hexafluorophosphate, quercetin hydrate, curcumin, Zn(II) phthalocyanine, eosin-Y disodium salt, psoralens and riboflavin arianor steel blue, tryptan blue, crystal violet, azure blue cert, azure B chloride, azure 2, azure A chloride, azure B tetrafluoroborate, thionin, azure A eosinate, azure B eosinate, azure mix sicc, and azure II eosinate, or a combination thereof. In some embodiments, the photosensitizer is methylene blue, Rose Bengal, tris(2,2′-bipyridine)ruthenium(II) hexafluorophosphate, quercetin hydrate, curcumin, Zn(II) Phthalocyanine, crystal Violet, azure-A, eosin-Y disodium salt, or a combination thereof. In some embodiments, the metallic nanoparticle-photosensitizer mixture is a zinc oxide-methylene blue mixture and the mixture is dispersed within the composition. In some embodiments, the metallic nanoparticle-photosensitizer mixture is a zinc oxide-Rose Bengal mixture.

In some embodiments, the high temperatures are temperatures of between 150° C. and 350° C. In some embodiments, the composition generates reactive oxygen species.

In some embodiments, the polymer is selected from the group consisting of polyvinyl chloride, polylactic acid, polybutylene succinate, ethylene-vinyl acetate, polyethylene, ultra-high-molecular-weight polyethylene, polypropylene, polystyrene, polyethylene terephthalate, polybutylene terephthalate, nylon, polymethyl methacrylate, polycarbonate, polyoxymethylene, acetal, thermoplastic polyurethane, silicone, high consistency rubber, styrene-ethylene-butylene-styrene, polyisoprene, polytetrafluoroethylene, fluorinated ethylene propylene, polyvinylidene fluoride, polybutylenesuccinate homopolymer, polybutylene adipate homopolymer, polybutyleneadipate-succinate copolymer, polyethylenesuccinate-adipate copolymer, polyethylene glycol succinate and polyethylene adipate homopolymer, poly(lactide), poly(glycolide), poly(lactide-co-glycolide), poly(L-lactide-co-trimethylene carbonate), poly(dioxanone), poly(butylene succinate), poly(butylene adipate), polyactic acid, poly(lactic-co-glycolic acid), polyglycolic acid, polycaprolactone, polydioxanone, polyanhydrides, polyorthoesters, polyether ketone, polysulfones, polyaryletherketones, polyethylene terephthalate based copolymers, liquid crystal copolymers, collagen, gelatin, chitosan, cellulose, cellulose acetate, carboxymethyl cellulose, hydroxypropyl methylcellulose, silk fibroin, alginate, and combinations thereof.

In another aspect, the present disclosure further relates to a method for manufacturing an antimicrobial polymer of claim 1 comprising the steps of: a) providing a mixture of a plurality of metallic nanoparticles and one or more photosensitizers to produce a metallic nanoparticle-photosensitizer mixture; b) combining a polymer with the metallic nanoparticle-photosensitizer mixture; c) heating the combined metallic nanoparticle-photosensitizer mixture and polymer to produce a molten mixture; and d) forming the molten mixture into a desired shape.

In some embodiments, the molten mixture is formed into a shape of a medical device by extrusion, injection molding, pouring, or a combination thereof. In some embodiments, the combined metallic nanoparticle-photosensitizer mixture is heated to between 150° C. and 350° C. In some embodiments, the combined metallic nanoparticle-photosensitizer mixture and polymer is heated within an extruder. In some embodiments, the method further comprises a step of transferring the molten mixture from an extruder barrel to a shaped cavity, wherein the shaped cavity forms the molten mixture into the desired shape. In some embodiments, the desired shape is the shape of a medical device.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTIONS OF THE DRAWINGS

The accompanying drawings, which are incorporated in and constitute a part of this specification, illustrate (one) several embodiment(s) of the invention and together with the description, serve to explain the principles of the invention.

FIG. 1 shows extruded materials with varying photosensitizers. From left to right, FIG. 1 shows: PCL (without any methylene blue, ZnO, or replacements), MB, Tris(2,2′-bipyridine)ruthenium(II) hexafluorophosphate, Quercetin hydrate, Curcumin, Zn(II) Phthalocyanine, Protoporphyrin, Crystal Violet, Azure-A, and Eosin Y disodium salt.

FIG. 2 shows extruded materials with varying metallic nanoparticles. From left to right, FIG. 2 shows: PCL (without any methylene blue, ZnO, or replacements), ZnO, silver nanoparticles, copper nanoparticles, cupric oxide nanoparticles, magnesium oxide nanoparticles, tungsten (VI) oxide nanoparticles, iron (II, III) oxide nanoparticles, and titanium dioxide nanoparticles.

FIG. 3 is an image of the polymer after extrusion with varying amounts of methylene blue. Methylene Blue concentration is % w/w from left to right: 0%, 0.05%, 0.1%, 0.25%, 0.5%, 1% 2%.

FIG. 4 is a bar graph showing the antimicrobial activity of extruded PLA samples without antimicrobial components and with antimicrobial components containing different % w/w of methylene blue against E. coli ATCC 8739.

FIG. 5 is an image of extruded polymers with no antimicrobial components (left), PLA with 2% w/w Rose Bengal and 2% w/w ZnO (middle), and PLA with 1% w/w Rose Bengal, 1% w/w methylene blue, and 1% w/w ZnO (right).

FIG. 6 is a bar graph showing the antimicrobial activity after 24 hours of exposure in light conditions of extruded PLA samples with and without antimicrobial components containing different sized ZnO nanoparticles against E. coli ATCC 8739.

FIG. 7 is a bar graph showing the antimicrobial activity after 24 hours of exposure in dark conditions of extruded PLA samples without antimicrobial components and with antimicrobial components containing different sized ZnO nanoparticles against E. coli ATCC 8739.

FIG. 8 is an image of the polymer after extrusion, retaining a deep blue color due to the dispersion of methylene blue.

FIG. 9 is a bar graph showing the antimicrobial activity of an extruded PCL sample with and without antimicrobial components against E. coli ATCC25922.

FIG. 10 is a graph showing tensile test data for a PCL control without MB/ZnO.

FIG. 11 is a graph showing tensile test data for a PCL sample containing MB/ZnO.

FIG. 12 shows silicone materials with varying photosensitizers. From left to right, FIG. 12 shows: silicone (without any methylene blue, ZnO, or replacements), RBL, Tris(2,2′-bipyridine)ruthenium(II) hexafluorophosphate, Quercetin hydrate, Curcumin, Zn(II) Phthalocyanine, Protoporphyrin, Crystal Violet, Azure-A, and Eosin Y disodium salt.

FIG. 13 shows silicone materials with varying metallic nanoparticles. From left to right, FIG. 13 shows: silicone (without any RBL, ZnO, or replacements), ZnO, silver nanoparticles, copper nanoparticles, cupric oxide nanoparticles, magnesium oxide nanoparticles, tungsten (VI) oxide nanoparticles, iron (II,III) oxide nanoparticles, and titanium dioxide nanoparticles.

FIG. 14 is an image of methylene blue/ZnO pour silicone samples prepared with different grinding times. From left to right, control with silicone only, 10 minutes of grinding, 15 minutes of grinding, 20 minutes of grinding.

FIG. 15 is an image of silicone prepared with and without Rose Bengal/ZnO mixture.

FIG. 16 is a graph showing tensile test data for a silicone control without RBL/ZnO.

FIG. 17 is a graph showing tensile test data for a silicone sample containing RBL/ZnO.

### DETAILED DESCRIPTION

The present invention may be understood more readily by reference to the following detailed description of preferred embodiments of the invention and the Examples included therein and to the Figures and their previous and following description.

### I. Definitions

To facilitate an understanding of the principles and features of the various embodiments of the disclosure, various illustrative embodiments are explained herein. Although exemplary embodiments of the disclosure are explained in detail, it is to be understood that other embodiments are contemplated. Accordingly, it is not intended that the disclosure is limited in its scope to the details of construction and arrangement of components set forth in the description or examples. The disclosure is capable of other embodiments and of being practiced or carried out in various ways.

In describing the exemplary embodiments, specific terminology will be resorted to for the sake of clarity. As used in the specification and the appended claims, the singular forms “a,” “an,” and “the” include plural references unless the context clearly dictates otherwise. For example, reference to a component is also intended to include the composition of a plurality of components. References to a composition containing “a” constituent are intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

As used herein, the term “dark conditions” means away from direct light sources. Dark conditions are those in which the antimicrobial composition is not exposed to an external light source sufficient to activate a photosensitizer via photonic excitation. Dark conditions may include environments with minimal or incidental ambient light and are not limited to complete darkness. As used herein, the term “light source” refers to a source of light such as sunlight, visible light, laser light, or any other light of any specific wavelength. The term ‘dark conditions” may be used interchangeably with the term “in the dark.”

As used herein, the term “photosensitizer” refers to a substance capable of absorbing light and transferring energy to desired reactants. Photosensitizer refers to a compound capable of generating reactive chemical species, including reactive oxygen species and/or free radicals, either upon exposure to a light source or, in some embodiments of the present invention, in the absence of light when combined with one or more metallic nanoparticles.

As used herein, the term “sterilization” refers to a method for sterilizing medical devices and/or pharmaceuticals. Sterilization refers to a process for substantially reducing or eliminating viable microorganisms from a medical device or composition, including but not limited to bacterial, viral, and fungal species.

As used herein, the term “subject,” “subjects,” “subjects in need thereof,” and “individuals” includes humans as well as non-human subjects. It will be understood that the subject to which a compound of the invention is administered need not suffer from a specific traumatic state. Indeed, the implants of the invention may be administered prophylactically. The terms “subject,” “subjects,” “subjects in need thereof,” and “individuals” can be used interchangeably herein.

As used herein, “embedded” refers to having one substance dispersed or suspended in another substance, for example, by mixing. In the antimicrobial composition, the antimicrobial nanoparticles are preferably evenly dispersed in the polymer.

As used herein, “antimicrobial nanoparticles” refers to nanoparticle substances comprised of an antimicrobial agent and a support agent. As used herein, “antimicrobial agent” refers to any substance exhibiting antimicrobial properties, including but not limited to: bactericidal effects, fungicidal effects, bacteriostatic effects, and fungistatic effects. The antimicrobial agent is preferably a metallic nanoparticle with a photosensitizer.

As used herein, “nanoparticles” refers to particles having a diameter in the range of 0.1 nm to 1000 nm. Preferably, the particles are less than 500 nm, more preferably less than 300 nm, and even more preferably less than 200 nm. Most preferably, the nanoparticles are less than or equal to 100 nm in diameter.

### II. Compositions

### A. Antimicrobial Polymer Mixture

### 1. Metallic Nanoparticles

Provided herein are antimicrobial polymer compositions for the production of antimicrobial medical devices. Also provided is a process of making antimicrobial medical devices from antimicrobial metallic nanoparticle-photosensitizer polymer mixtures. In some embodiments, the process involves the incorporation of a ROS producing compound and a polymer into an antimicrobial medical device.

In some embodiments, ROS-producing compounds used herein may be selected from, but are not limited to, a group of metallic nanoparticles and/or photosensitizers. In some embodiments, the process involves the incorporation of a nanoparticle, in this case zinc oxide nanopowder, and a photosensitizer dye, methylene blue in this case, and a polymer into an antimicrobial medical device. In some embodiments, the zinc oxide nanopowder particle size is selected from 10 nm, 20 nm, 30 nm, 40 nm, 50 nm, 60 nm, 70 nm, 80 nm, 90 nm, or 100 nm. In one embodiment, the particle size is 20 nm. In another embodiment, the zinc oxide nanopowder particle size is less than 50 nm. In yet another embodiment, the zinc oxide nanopowder particle size is 100 nm.

Polymer materials used herein may be selected from a group consisting of commodity and engineering thermoplastics (Polyvinyl chloride, polyethylene, ultra-high-molecular-weight polyethylene, polypropylene, polystyrene, polyethylene terephthalate, polybutylene terephthalate, nylon, polymethyl methacrylate, polycarbonate, polyoxymethylene, acetal), thermoplastic elastomers (thermoplastic polyurethane, silicone, high consistency rubber, styrene-ethylene-butylene-styrene, polyisoprene, fluoropolymers (polytetrafluoroethylene, fluorinated ethylene propylene, polyvinylidene fluoride), thermoplastic aliphatic polyester (polybutylenesuccinate homopolymer, polybutylene adipate homopolymer, polybutyleneadipate-succinate copolymer, polyethylenesuccinate-adipate copolymer, polyethylene glycol succinate and polyethylene adipate homopolymer, poly(lactide), poly(glycolide), poly(lactide-co-glycolide), poly(L-lactide-co-trimethylene carbonate), poly(dioxanone), poly(butylene succinate), poly(butylene adipate)), bioabsorbable polymers (polyactic acid, poly(lactic-co-glycolic acid)), polyglycolic acid, polycaprolactone, polydioxanone, polyanhydrides, polyorthoesters), specialty high performance polymers (polyether ketone, polysulfones, polyaryletherketones, poly ethylene terephthalate based copolymers (e.g. Hyrel), liquid crystal copolymers), natural polymers (collagen, gelatin, chitosan, cellulose and cellulose derivatives (e.g. cellulose acetate, carboxymethyl cellulose, hydroxypropyl methylcellulose) silk fibroin, alginate), and mixtures thereof.

The term “nanoparticles” or “metallic nanoparticles” as used herein, refers to nanoparticles comprising a metal, metal oxide, alloy, or combinations thereof. In some embodiments, the metallic nanoparticles have at least one dimension in the range of about 1 to about 200 nm. In some embodiments, the zinc oxide nanopowder particle size is selected from 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, or 200 nm. In one embodiment, the particle size is 20 nm. In another embodiment, the zinc oxide nanopowder particle size is less than 50 nm. In yet another embodiment, the zinc oxide nanopowder particle size is 100 nm. In another embodiment, the zinc oxide nanoparticle size is 200 nm.

Nanoparticles typically, but not exclusively, comprise metals. They may also comprise alloys of two or more metals, or more complex structures such as core-shell particles, rods, stars, spheres, or sheets. A core-shell particle may typically comprise a core of one substance, such as a metal, metal oxide, or silica, surrounded by a shell of another substance, such as a metal, metal oxide, or metal selenide. The term “metallic” as used herein is intended to encompass all such structures having a metallic outer surface.

In some embodiments, the outer surface of the metallic nanoparticles of the present invention comprises a main group metal or transition metal, such as cobalt. In other embodiments, the metallic nanoparticles comprise one or more of zinc, gold, silver, and/or copper nanoparticles, and/or alloys of two or more of these metals. Metallic nanoparticles suitable for use in the present compositions may include, but are not limited to, nanoparticles comprising zinc, zinc oxide, silver, copper, cupric oxide, magnesium oxide, iron (II,III) oxide, tungsten (VI) oxide, titanium dioxide, graphene oxide, gold, alloys thereof, or combinations thereof. In some embodiments, the nanoparticles are zinc oxide nanoparticles. In some embodiments, the zinc oxide nanoparticles react with triplet oxygen to form a singlet oxygen and/or free radicals. In some embodiments, the zinc oxide generates both singlet oxygen and free radicals.

In some embodiments, the metallic nanoparticles exhibit antimicrobial activity. In some embodiments, the metallic nanoparticles are selected such that they exhibit antimicrobial activity. In some embodiments, when combined with one or more appropriately-selected photosensitizers, as described herein, the combination of the metallic nanoparticles and the photosensitizers generates antimicrobial activity. In some embodiments, the antimicrobial activity of the combination is greater than the antimicrobial activity exhibited by the metallic nanoparticles or the photosensitizers alone.

In some embodiments, the metallic nanoparticles of the present invention are zinc oxide nanoparticles. In some embodiments, the zinc oxide activates the photosensitizer in the mixture to react with triplet oxygen to form a singlet oxygen and/or free radicals. In some embodiments, the mixture generates both singlet oxygen and free radicals.

Singlet oxygen generation may be measured by assay: several such methods are known to those skilled in the art, for example, photoluminescence. Free radical generation may be measured using electron proton resonance (EPR).

Examples of metallic nanoparticles that may be suitable are nanoparticles having a diameter greater than about 2 nm, which exhibit plasmon resonance in the wavelength band of about 200 to about 1600 nm, i.e., covering the visible to near-infrared bands. The plasmon resonance may be measured by UV spectroscopy. In some embodiments, nanoparticles will exhibit plasmon resonance at wavelengths of from about 500 to about 600 nm.

Another property that may be used to help select a suitable nanoparticle is its catalytic effect on the photosensitizer. When activated, the extinction coefficient may be enhanced compared to the photosensitizer in the presence of the nanoparticle. The extinction coefficient could be measured using a spectrophotometer. Any enhancement in the extinction coefficient in the presence of a nanoparticle is acceptable. In some embodiments, the extinction coefficient may range anywhere from about 2 to about 30 times or more; in others, it may range from about 5 to about 30 times or more; in others, it may range from about 10 to about 30 times or more, and in others, it may range from about 20 to about 30 times or more, compared to what is expected based on the same concentration of the photosensitizer not in the presence of a nanoparticle.

In some embodiments, the outer surface of the nanoparticles may comprise one or more of the group comprising zinc, gold, silver or copper. In some embodiments, the nanoparticles may comprise one or more of the group comprising zinc or alloys thereof. Suitable alloys may also comprise one or more other metals, such as silver, copper, gold, aluminum, or combinations thereof.

In other embodiments, the nanoparticles described in the preceding paragraph comprise core-shell particles. It is possible for such core-shell particles to comprise a magnetic core or magnetic layer. An example of such a magnetic core-shell particle is a particle having a magnetic core and an outer shell that comprises gold. In some embodiments, the nanoparticles are zinc oxide nanoparticles.

### 2. Antimicrobial Photosensitizers

A photosensitizer is a compound that can be excited by light of a specific wavelength. Thus, such a compound may have an absorption band in the ultraviolet, visible or infrared portion of the electromagnetic spectrum and, when the compound absorbs radiation within that band, it generates cytotoxic species, thereby exerting an antimicrobial effect. The effect may be due to the generation of singlet oxygen, but the invention is not limited to photosensitizers that exhibit antimicrobial effects through generation of singlet oxygen.

In some embodiments, the photosensitizer is chosen such that it generates a singlet oxygen and/or free radicals, and/or the selected nanoparticle acts as a catalyst to activate the photosensitizer, generating singlet oxygen and/or free radicals. Preferably, the nanoparticle photosensitizer mixture generates both singlet oxygen and free radicals without the presence of a light source. Singlet oxygen and free radical generation may be measured as described above. In some embodiments, the photosensitizer exhibits antimicrobial activity.

Without wishing to be bound by theory, it is thought that the photosensitizer and nanoparticles are associated via dative covalent bonds, wherein the electrons are provided by, for example, S or N moieties on the photosensitizer.

In some embodiments, the photosensitizer demonstrates antimicrobial activity within a subject's body without the need to be activated by a light source. In some embodiments, the photosensitizer is one or more photosensitizers selected from the group comprising: phenothiaziniums (e.g. toluidine blue O, methylene blue, dimethylmethylene blue), phthalocyanines (e.g. zinc, silicon and aluminum phthalocyanines), halogenated xanthenes (e.g. Rose Bengal), porphyrins (e.g. haematoporphyrin derivatives, deuteroporphyrin), chlorins (e.g. tin chlorin e6, poly-lysine derivatives of tin chlorin e6, m-tetrahydroxyphenyl chlorin, benzoporphyrin derivatives, tin etiopurpurin), bacteriochlorins, phenazines (e.g. neutral red), acridines (e.g. acriflavine, proflavin, acridine orange, aminacrine), texaphyrins, cyanines (e.g. merocyanine 540), anthracyclins (e.g. adriamycin and epirubicin), ruthenium-based organometallics (e.g. Tris(bipyridine)ruthenium(II), cis-Bis(isothiocyanato)bis(2,2′-bipyridyl-4,4′-dicarboxylato) ruthenium (II), and TLD-1443), iridium-based organometallics (e.g. Ir(ppy)₃(fac-Tris(2-phenylpyridine) iridium (III)), [Ir(ppy)₂(bpy)]⁺), pheophorbides, sapphyrins, fullerene, perylenequinonoid pigments (e.g. hypericin, hypocrellin), gilvocarcins, terthiophenes, benzophenanthridines, psoralens and riboflavin arianor steel blue, tryptan blue, crystal violet, azure blue cert, azure B chloride, azure 2, azure A chloride, azure B tetrafluoroborate, thionin, azure A eosinate, azure B eosinate, azure mix sicc, and azure II eosinate. In particular embodiments, the photosensitizers are methylene blue or Rose Bengal.

a. Organic Photosensitizers

The photosensitizers may be organic (including dyes, porphyrins, chlorins, furocoularins, xanthenes or monoterpene) or non-organic (including gaseous mercury, quantum dots, or nanowires/nanorods).

Amongst the list of organic photosensitizers, three main families with similar mechanisms can be identified.

i. Porphyrins and Chlorins.

In some embodiments, the photosensitizer is categorized as a porphyrin or chlorin photosensitizer. These photosensitizers include porphyrin, porfimer, temoprfin, and talaporfin sodium. Porphyrin and porphyrin-like molecules constitute the most common type of photosensitizer; they are all built around the porphyrin center:

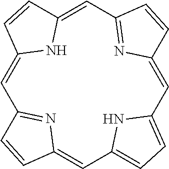 [SMILES: C1=Cc2cc3ccc(cc4nc(cc5ccc(cc1n2)[nH]5)C=C4)[nH]3]

This molecule, thanks to its highly conjugated structure, can absorb photonic energy to enter an excited state commonly referred to as Sn (where n is the level of excitation, n=0 being the ground state). Each level of energy is stored in the higher molecular orbitals (moving from HOMO to LUMO) of the molecule and can be transferred to the orbital of triplet oxygen to excite it to a singlet state. Singlet oxygen is highly reactive to organic species and promotes the formation of peroxo bridges, which in turn promote the formation of radicals, also known as ROS (Reactive oxygen species).

The absorption of photons by the porphyrin is determined by the energy bandgap between the HOMO and LUMO orbitals. Various strategies can be developed to affect that bandgap, mostly by modifying the π-conjugated network and adding or replacing some heteroatoms. The resulting effect can determine the energy of the photon that is necessary to induce an excited state of porphyrin.

One strategy that can be employed is the addition of a metal-based core to the center of the porphyrin. This is commonly referred to as metalloporphyrin and is largely inspired by the structure of hemes in hemoglobin. These molecules are generally synthesized ex situ but could be formed in situ under the right conditions.

It has been observed that under certain conditions, porphyrin could reach an excited state, not through the direct absorption of a photon but through the transfer of excitement via another molecule (enzymes, protein) (Afonso S G, Enriquez de Salamanca R, Batlle A M. Braz J Med Biol Res. 1999 March; 32(3): 255-66).

Therefore, it is possible that a cooperative effect between the porphyrin (or porphyrin-like compound) and a metal nanoparticle could, under the right circumstances, catalyze the formation of ROS without the need for photon absorption.

ii. Dyes and Xanthenes

In some embodiments, the photosensitizer is categorized as a dye and xanthenes photosensitizer. These photosensitizers include acridine orange, methylene blue, toludine blue O, Rose Bengal, and erythrosine. This family presents a structure that conjugates π bonds around a benzenic center. In its oxidized state, the dye's benzenic ring (one or more) is not saturated and instead displays a 1,4-cyclohexadiene structure linked to double bonds in positions 3,6. Those double bonds can be linked directly or via conjugation to a heteroatom presenting non-binding doublets (e.g., O, N, S). The driving mechanism for the reduction is the completion of the benzene ring and its stabilization via aromaticity. This process is initiated by the loss of two (or more) electrons and the bonding of one or more of the terminal heteroatoms to a hydrogen atom from the environment surrounding the dye. This transformation ends with the more thermodynamically stable reduced form of the dye.

This process is entirely reversible should the reduced dye be exposed to a strong oxidizer (more thermodynamically reactive than the oxidized dye) and the dye being in a medium allowing proton exchange (e.g., water). This applies directly to the examples disclosed herein with methylene blue. Along with the aromatization of a benzene ring, the heteroatom can be involved in the closing (during the reduction) of a heterocycle. This applies directly to the examples disclosed herein with Rose Bengal (Hai, Yue & Feng, Shan & Wang, Lili & Ma, Yetao & Zhai, Yiran & Wu, Zijun & Zhang, Sichao & He, Xin. (2017). Coordination Mechanism and Bio-Evidence: Reactive γ-Ketoenal Intermediated Hepatotoxicity of Psoralen and Isopsoralen Based on Computer Approach and Bioassay. Molecules. 22. 1451. 10.3390/molecules22091451).

Because of this highly conjugated structure, the band gap between HOMO and LUMO is such that the molecule can absorb a photon and enter an excited state. This energy can then be transferred to molecular oxygen to transform it from triplet oxygen to singlet oxygen, leading to the creation of ROS.

However, it should also be mentioned that, because of their natural oxidative properties (and their reversible nature), this family of molecules can demonstrate antibacterial activity in the absence of a photon.

iii. Organometallic Photosensitizers

In some embodiments, the photosensitizer is categorized as an organometallic photosensitizer. Organometallic photosensitizers contain a metal atom chelated to at least one organic ligand. Organometallic photosensitizers commonly used are ruthenium, rhodium, and iridium-based compounds. These photosensitizers include Tris(bipyridine)ruthenium(II), cis-Bis(isothiocyanato)bis(2,2′-bipyridyl-4,4′-dicarboxylato) ruthenium (II), TLD-1443), Ir(ppy)₃(fac-Tris(2-phenylpyridine) iridium (III)), and [Ir(ppy)₂(bpy)]⁺. These transition metals have highly filled d-orbitals, allowing them to provide electrons to ligands, which are often I-conjugated. The interaction between the metal and ligand leads to a large continuum of orbitals where the highest occupied molecular orbital (HOMO) is metal-centered, and lowest unoccupied molecular orbital (LUMO) is ligand-based, allowing for excited electrons to switch multiplicities via intersystem crossing and generation of cytotoxic ROS.

iv. Exotic Photosensitizers

In some embodiments, the photosensitizer is categorized as an exotic photosensitizer. These photosensitizers include azulene and psoralen. This family is less defined and regroups mostly seldomly used photosensitizers. These molecules usually bind to specific sites upon photon absorption and are therefore non-catalytic and lack a specific mechanism. Based on their undefined properties, this category is not included in the disclosed compositions and methods. The photosensitizer categories that are used in the disclosed compositions and methods are: Porphyrins, Chlorins, Dyes, Xanthenes, and organometallics. Other photosensitizers can be listed and added individually based on their structure and mechanism of action.

The proportion of metallic nanoparticle: photosensitizer may vary. Typically, the nanoparticle comprises many atoms; the number of photosensitizer molecules interacting with each nanoparticle may also vary.

The metallic nanoparticle-photosensitizer mixture may also comprise further components. In some embodiments, it may have a targeting moiety associated with it. The targeting moiety can be associated with the conjugate via any suitable means, for example, it may be attached to the nanoparticle core or to the photosensitizer. Such targeting moieties may be suitable, for example, for targeting specific microorganisms or for targeting cancer cells. For example, they may be antibodies with specificity for the target organism or cancer cell. Other examples of targeting moieties include bacteriophages, protein A (targets Staphylococcus aureus), and bacterial cell wall-binding proteins or peptides.

### 3. Polymers

It is a requirement of the present invention that the polymer be chosen such that it can be formed into an implantable medical device. In some embodiments, the polymer is selected from the group consisting of commodity and engineering thermoplastics (Polyvinyl chloride, polyethylene, ultra-high-molecular-weight polyethylene, polypropylene, polystyrene, polyethylene terephthalate, polybutylene terephthalate, nylon, polymethyl methacrylate, polycarbonate, polyoxymethylene, acetal), thermoplastic elastomers (thermoplastic polyurethane, silicone, high consistency rubber, styrene-ethylene-butylene-styrene, polyisoprene, fluoropolymers (polytetrafluoroethylene, fluorinated ethylene propylene, polyvinylidene fluoride), thermoplastic aliphatic polyester (polybutylenesuccinate homopolymer, polybutylene adipate homopolymer, polybutyleneadipate-succinate copolymer, polyethylenesuccinate-adipate copolymer, polyethylene glycol succinate and polyethylene adipate homopolymer, poly(lactide), poly(glycolide), poly(lactide-co-glycolide), poly(L-lactide-co-trimethylene carbonate), poly(dioxanone), poly(butylene succinate), poly(butylene adipate)), bioabsorbable polymers (polyactic acid, poly(lactic-co-glycolic acid)), polyglycolic acid, polycaprolactone, polydioxanone, polyanhydrides, polyorthoesters), specialty high performance polymers (polyether ketone, polysulfones, polyaryletherketones, poly ethylene terephthalate based copolymers (e.g. Hyrel), liquid crystal copolymers), natural polymers (collagen, gelatin, chitosan, cellulose and cellulose derivatives (e.g. cellulose acetate, carboxymethyl cellulose, hydroxypropyl methylcellulose) silk fibroin, alginate), and mixtures thereof.

In some embodiments, the polymer is a thermoplastic polyester. Thermoplastic polyesters are products of aromatic dicarboxylic acids and aliphatic diols. Aliphatic polyesters are derived from naturally occurring compounds such as lactide and glycolide. Aliphatic polyesters are an important class of biocompatible and biodegradable materials (Chesterman J, et al. Principles of Tissue Engineering 5: 317342 (2020)). Aliphatic polyesters are commonly used in biomedical applications due to their nontoxic properties and their products. In certain embodiments, the polymer is polyactic acid.

In some embodiments, the novel ROS-producing compounds are polymer compositions that have been found to exhibit particularly effective antimicrobial properties. Thus, all uses of mixtures as described herein apply to the novel compositions.

### B. Metallic Oxides and Methylene Blue Mixtures

Provided herein are antimicrobial compositions comprising metallic oxides and methylene blue mixtures.

Zinc oxide (ZnO) is an inorganic compound that typically can be found in the form of a powder. ZnO can be directly added to the nanoparticle-photosensitizer polymer mixture for extrusion or silicone molding/injection. Zinc oxide is known to generate reactive oxygen species (Guo, et al., Toxicol In Vitro, 27 (2): 731-738 (2013)). Zinc ions do not leach out when ZnO is not in water. If submerged in water and at an acidic pH, it may begin to leach Zn+ ions.

Complexes of methylene blue with the metal form of precious metals (most likely nanoparticles) can be favored due to the availability of the S doublet (divalent sulfur). That includes affinity for Ag or Au, and potentially other thiophilic compounds.

In some embodiments, it is possible that the beneficial effect of the addition of both a metallic nanoparticle and a photosensitizer relies on the different mechanisms of action, the photosensitisers through ROS (oxygen radicals) formation leading to cell/DNA oxidation, the metal nanoparticles through membrane accumulation/permeation leading to apoptosis or through reactive oxygen species generation (Bruna T, Maldonado-Bravo F, Jara P, Caro N. Int J Mol Sci. 2021 Jul. 4; 22 (13): 7202). Silver nanoparticles exhibit ion release, or leaching, which provide their method for killing microorganisms.

Additional metallic oxides suitable for use in such mixtures include, but are not limited to, magnesium oxide, cupric oxide, iron oxide, tungsten oxide, titanium dioxide, and combinations thereof. Similarly, copper, cupric oxide, and magnesium oxide can be directly added to the nanoparticle-photosensitizer polymer mixture for extrusion or silicone molding/injection but exhibit ion release. Iron oxide nanoparticles can release ions at acidic pH but not at a pH of about 7.

The following nanoparticles can be directly added to the nanoparticle-photosensitizer polymer mixture for extrusion or silicone molding/injection and exhibit reactive oxygen species release: Tungsten (VI) (Popov, et al., Mater. Sci. Eng. C. Mater. Biol. Appl. 108:110494 (2020)), graphene oxide, and titanium dioxide.

### 1. Mechanisms of Reactivity of Methylene Blue in the Dark

In the presence of light, photons with a specific energy level can activate methylene blue (MB). Once activated MB can react with “classic” oxygen, also called triplet oxygen (³O₂), to form singlet oxygen (¹O₂). The difference between these 2 types of oxygen lies in the outer electron shell configuration.

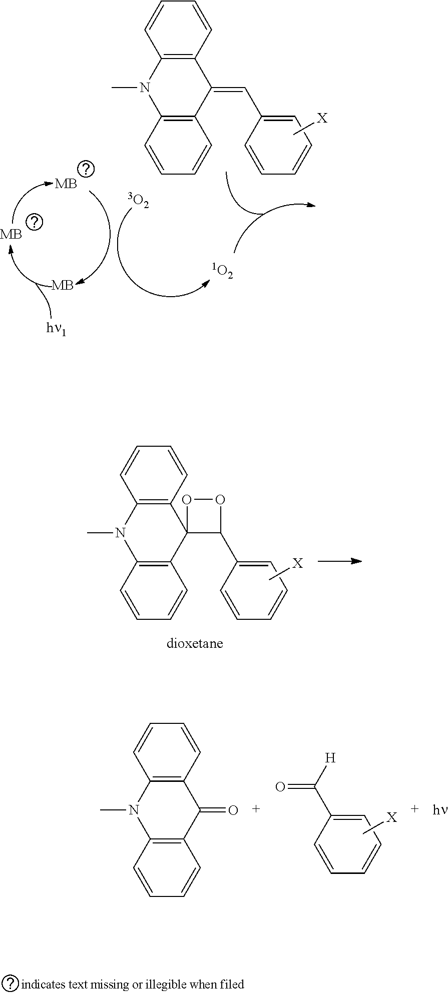

Having an empty orbital results in increased reactivity of singlet oxygen to facilitate oxidation steps, such as the oxidation of lipids or proteins that constitute the bacterial cell wall and lead to their destruction.

In some embodiments, MB acts as a catalyst and is regenerated under its oxidated form (blue).

In the example below, we can see the oxidation by singlet oxygen of a C—C bond in dioxethane leading to its separation in 2 units along with the catalytic cycle of MB (Lee, J. Bioluminescence, the Nature of the Light, (2020) University of Georgia).

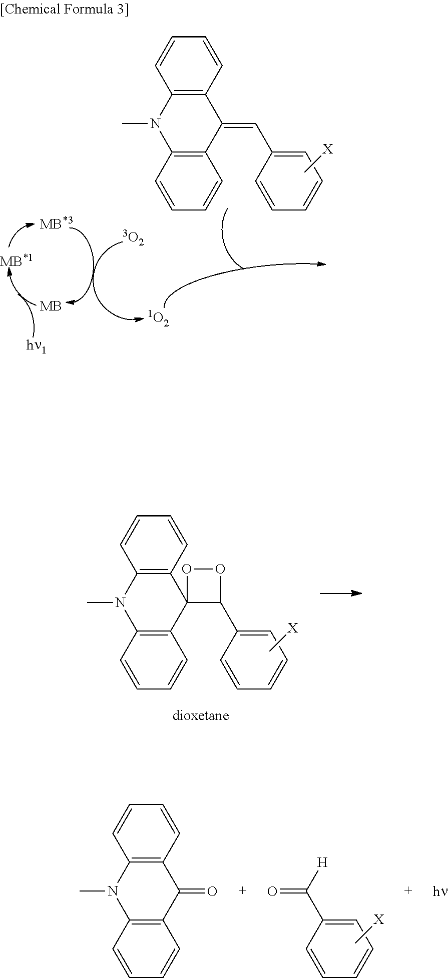

In the dark, the absence of photons means there is no basis for the catalytic cycle of the MB and therefore no formation of singlet oxygen. However, under biological conditions, the reaction of the MB in its oxidized form (blue) with organic species (such as bacterial cell walls) is catalyzed, the source of electrons and protons being the bacteria themselves. This reaction, however, is not reversible and will progressively lead to the reduction of the MB to leucomethylene blue (LMB) (reduced form, colorless) (Mahlum, J D et al., J. Phys. Chem. C 2021, 125, 17, 9038-9049).

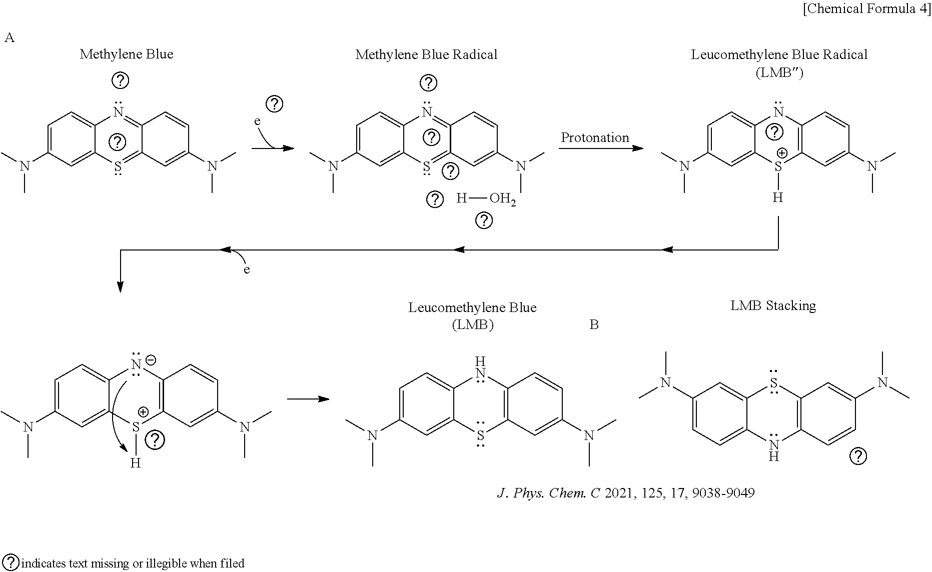

It is expected, that the MB would progressively become inactive as it is reduced, but this is not observed visually (the reduction from blue to colorless) in practice when samples are exposed to bacteria in light conditions. Depending on the concentration encapsulated, the time to inactivity could vary (based on flow rate and exposure to reducing agents, such as bacteria). As such high concentrations are encapsulated, it is expected this is why this reduction in activity has not been observed in experiments. However, there is a possibility that dissolved oxygen could lead to the oxidation of LMB even without light activation. This can be observed in the famous blue bottle experiment (The ‘blue bottle’ experiment in association with the Nuffield Foundation).

Other photosensitizers which provide light-dependent reactive oxygen species production include Tris(2,2′-bipyridine)ruthenium(II) hexafluorophosphate, quercetin hydrate, curcumin, Zn(II) phthalocyanine, crystal violet, azure-A, and eosin-Y.

### C. Polymers

In some embodiments, the polymer comprises a structure, such as a matrix structure. As used herein, a “polymer matrix structure” refers to, for example, the repeating structural unit of the polymer, which may include cross-linking. The polymer is configured such that the antimicrobial ions are able to freely permeate or migrate through the polymer and to the surface of the composition to maintain or achieve a sufficient or effective amount of the antimicrobial agent on the surface of the composition. In particular embodiments, the polymer comprises spaces or pathways that are of sufficient size to allow the ionic metal nanoparticles to migrate or pass through to the surface of the composition. In further embodiments, the polymer may comprise charged or partially charged groups in the spaces or pathways providing migrating ions a stabilizing structure for the ions to move through the body of the polymer. In other embodiments, the polymer comprises a chemical gradient or a chemical potential gradient to drive the migration of the ionic antimicrobial agents.

Polymers having low crystallinity, high quality, and also lacking impurities that may reflect or absorb light, are preferred. For example, polymers are selected for use in an antimicrobial composition based on low crystallinity, appropriate density, presence or absence of charged functional groups, chain length, and/or branch structure. For example, using polymers having less than 75% crystallinity, less than 50% crystallinity, less than 25% crystallinity, less than 10% crystallinity, less than 5% crystallinity, less than 3% crystallinity, less than 2% crystallinity, less than 1% crystallinity, or less than 0.5% crystallinity.

Examples of polymers include, but are not limited to: polycarbonate (PC), polyether block amide (PEBA), acrylic polymer, polyolefin polymer, thermoplastic elastomers including thermoplastic styrenic elastomer (TPE-S), thermoplastic polyester elastomer (TPE-E), thermoplastic polyolefin olefin (TPE-O), thermoplastic vulcanizate (TPE-V), thermoplastic vulcaniztes, thermoplastic synthetic polymers, polyethylenes, polypropylene, polypropylene copolymers, polyaryletherketones, polyether ether ketone, polyether ketone ketone, polyether ketone, polyethylene terephthalate, polybutylene terephthalate acetate polymers, ethylene-vinyl acetate, polystyrene, polyvinyl chloride, polyvinylidene chloride, polyamides, polyether block amides, polyacetals, polyesters, polyvinyl alcohols, polyvinylpyrrolidone blends, carbonate polymers, butyrate polymer, halogenated polymer, polyether polymer, styrene butadiene polymer, polyurethanes, polysulfone, polyethersulfone, polyphenylsulfone, liquid silicone rubber and silicone elastomers, silicone oil, silicone resin, ABS resins, acrylic resins, fluorine-containing resins, polyurethane elastomers, polyethylene naphthalate, polyetherimide, polyimides, hydrogel-forming polymers, polyethylene variants including low-density polyethylene (LDPE), high-density polyethylene (HDPE), linear low-density polyethylene (LLDPE), and ultra high molecular weight polyethylene (UHMWPE), cyclic olefin polymers (COP), cyclic olefin copolymers (COC), polyvinylidene fluoride (PVDF), hydrogel-forming polymers including poly(2-hydroxyethyl methacrylate (PHEMA), poly(ethylene glycol) diacrylate (PEGDA), and polyvinyl alcohol (PVA) hydrogels, or polyester elastomers.

In some embodiments, antimicrobial nanoparticles, photosensitizers, or mixtures thereof provide antimicrobial activity at the surface of a polymer. In some embodiments, the antimicrobial nanoparticles, photosensitizers, or mixtures thereof are embedded within the polymer matrix and provide antimicrobial activity to the polymer matrix and to the surface of the polymer. In some embodiments, the antimicrobial nanoparticles, photosensitizers, or mixtures thereof are embedded within the polymer, coated upon the polymer, or a combination thereof, and provide antimicrobial activity to the polymer matrix and to the surface of the polymer. In some embodiments, the antimicrobial nanoparticles, photosensitizers, or mixtures thereof embedded within the polymer matrix may provide antimicrobial activity to the polymer surface over an extended period of time.

Some advantageous properties of the above-listed polymers include low haze but improved tactile acceptability, specific chemical resistance, reduced UV degradation, reduced age-related crystallization, and improved impact absorption. According to some embodiments, the material of the present invention comprises a polymer selected from a synthetic polymer, a naturally occurring polymer, or combinations thereof. Each possibility represents a separate embodiment of the invention. According to some embodiments, the synthetic polymer is selected from the group consisting of organic polymers, inorganic polymers, and bioplastics. In further embodiments, the polymer is selected from the group consisting of polyamide, polyester, polyvinyl chloride, acrylic, polyalkene, polysiloxane, nylon, nitrile, silicone, fluoropolymers, polyvinyl acetate, starch-based polymer, cellulose-based polymer, dispersions, and mixtures thereof. According to some currently preferred embodiments, the polymer is selected from polyester, polyalkene, and polyamide. The polyalkene may be selected from the group consisting of polypropylene, polyethylene, polybutylene and combinations thereof. Each possibility represents a separate embodiment of the invention. The polymer may be water-based or solvent-based.

### D. Antimicrobial Products

The antimicrobial polymer compositions or formulations disclosed herein are intended for incorporation into a wide range of products in which sustained antimicrobial activity is desirable. In particular, the compositions are suited for products exposed to microbial contamination during use and that may operate in both light and dark environments, including prolonged in vivo use. The antimicrobial activity may be provided through antimicrobial nanoparticles, photosensitizers, or combinations thereof embedded within, coated upon, or otherwise incorporated into polymeric materials.

### 1. Implantable and Insertable Medical Devices

In some embodiments, the antimicrobial polymer compositions are used to manufacture implantable or insertable medical devices. Such devices are particularly susceptible to microbial colonization and biofilm formation and often operate entirely under dark conditions. Non-limiting examples include: catheters (including urinary, intravascular, central venous, peritoneal, and drainage catheters), stents (vascular, biliary, ureteral, gastrointestinal), endotracheal tubes and airway management devices, implantable tubing, shunts, and conduits, surgical meshes and tissue reinforcement materials, sutures, staples, clips, and wound-closure devices, orthopedic implants, spacers, anchors, and fixation devices, dental implants, orthodontic appliances, and aligners, implantable pumps, reservoirs, and drug-delivery devices, implantable sensors, leads, insulation jackets, and medical electronics housings. In certain embodiments, the antimicrobial composition is embedded throughout the polymer matrix of the device to provide both surface and bulk antimicrobial activity over extended periods.

### 2. Wound Care and Skin-Contact Medical Products

In some embodiments, the antimicrobial polymer compositions are incorporated into wound care and skin-contact products, where control of microbial load is critical to healing. Non-limiting examples include: wound dressings, foams, films, and hydrogels, -silicone scar therapy sheets and post-surgical dressings, adhesive bandages and medical tapes, burn dressings and temporary skin substitutes, negative pressure wound therapy (NPWT) components, stomy barriers, seals, and accessories. These products may be configured to maintain antimicrobial activity during prolonged contact with skin or wound exudate.

### 3. Respiratory, Urological, and Gastrointestinal Products

In some embodiments, the antimicrobial polymer compositions are used in devices that contact respiratory, urinary, or gastrointestinal systems, including: ventilator circuits, breathing tubes, and connectors, oxygen masks, cannulas, and nebulizer components, foley catheter balloons, valves, and drainage connectors, feeding tubes (PEG, NG, NJ), rectal tubes, and fecal management systems, colostomy and ileostomy components. Such products benefit from antimicrobial activity in moist, nutrient-rich environments that promote microbial growth.

### 4. Diagnostic, Laboratory, and Healthcare Consumables

In some embodiments, the antimicrobial polymer compositions are incorporated into single-use or reusable medical and laboratory products, including: petri dishes, culture plates, and laboratory containers, sample collection tubes, syringes, and specimen cups, pipette tips, pipettes, and microfluidic devices, swab shafts and diagnostic testing components. These products may reduce cross-contamination and improve hygiene in clinical and laboratory settings.

### 5. Hospital, Clinical, and Environmental Surfaces

In some embodiments, the antimicrobial polymer compositions are used in hospital and clinical environment products, including: bed rail covers, mattress covers, and cushions, medical instrument trays and procedure mats, IV pole grips, handles, and high-touch surfaces, reusable tourniquets and wearable patient monitors, privacy curtains, drapes, and protective coverings. Such products can assist in reducing healthcare-associated and nosocomial infections.

### 6. Consumer, Wearable, and Personal Care Products

In some embodiments, the antimicrobial polymer compositions are incorporated into consumer or wearable products, including: reusable face masks and respirator shells, hearing aids, earbuds, and wearable device components, dentures, retainers, mouthguards, and dental appliances, smartwatch bands and fitness wearable housings, contact lens cases and personal hygiene accessories.

### 7. Veterinary, Agricultural, and Animal Health Products

In some embodiments, the antimicrobial polymer compositions are used in veterinary and animal health applications, including: veterinary catheters and wound dressings, implantable animal devices, livestock equipment liners and tubing, feeding and watering components

### 8. Non-Medical and Industrial Products

In further embodiments, the antimicrobial polymer compositions are incorporated into non-medical polymeric articles, including: food and beverage processing components, packaging, containers, and wraps, water filtration housings and membranes, consumer goods requiring antimicrobial durability.

### 9. Textile, Fiber, and Fabric-Based Products

In some embodiments, the antimicrobial polymer compositions are incorporated into fibers, yarns, textiles, and fabrics, including woven, knitted, or nonwoven textiles; medical gowns; drapes; linens and bedding; compression garments; wraps and bandages; and filter media for masks or air purification systems. The antimicrobial compositions may be distributed uniformly within fibers or coated onto textile surfaces.

According to some embodiments, the antimicrobial product is a textile product or a non-textile polymeric article. According to some embodiments, the yarn, textile, or fabric is formed into the textile product. According to some embodiments, the textile product comprises an extruded polymer. According to certain embodiments, the fiber comprises an extruded polymer. According to additional embodiments, the non-textile polymeric article comprises an extruded, molded, or cast polymer.

According to some embodiments, the antimicrobial product comprises a fiber, a yarn, a textile, a fabric, a film, or a foil. Each possibility represents a separate embodiment of the invention. The textile can be selected from a woven textile, a knit textile, a non-woven textile, a needle-punch textile, or felt. Each possibility represents a separate embodiment of the invention. According to certain embodiments, the semi-final product is a fiber. The fiber can be a filament fiber or a staple fiber. According to some embodiments, the antimicrobial compositions are incorporated substantially uniformly within the fiber. The fiber can be formed into a yarn, textile, or fabric.

According to some embodiments, the fiber is a polymeric fiber, being synthetic or semi-synthetic. According to some embodiments, the material further comprises a natural fiber. Thus, in some embodiments, the fiber is a blend of the polymeric fiber with a natural fiber.

According to further embodiments, the material comprises a natural fiber in a weight percentage of up to about 85% of the total weight of the material. In particular embodiments, the material comprises a natural fiber in a weight percentage of up to about 70% of the total weight of the textile product. The natural fiber may be selected from the group consisting of cotton, silk, wool, linen, and combinations thereof. Each possibility represents a separate embodiment of the invention. In a certain embodiment, the material comprises cotton.

According to some embodiments, the material comprises a blend of a polymeric fiber and a natural fiber. In certain such embodiments, the combined weight of the antimicrobial composition constitutes from about 0.25% to about 5% wt. of the total weight of the material.

According to some embodiments, the material is in the form of a textile product or a non-textile polymeric article. Each possibility represents a separate embodiment of the invention. The textile product may be selected from clothing items, bedding textiles, laboratory or hospital textiles, medical textiles (including bandages or sutures), and textiles for internal use, as well as personal hygiene articles. The non-limiting examples of the textile products include pillowcases, eye-masks, gloves, socks, stockings, sleeves, shoe covers, slippers, undergarments, industrial uniforms, sportswear, towels, kitchen cloths, lab coats, floor cloths, sheets, bedding, curtains, textile covers, hard surface covers, diapers, incontinence pads, feminine hygiene products, gauze pads, monolithic extruded membranes, body-suits, trans-dermal patches, bandages, adhesive bandages, sutures, sheaths and textiles for internal use.

The non-textile polymeric article may be selected from packaging or wrapping materials, laboratory equipment, hospital equipment (preferably disposable hospital equipment), birth-control devices, agricultural products, covers for consumer items, and sanitary products. The non-limiting examples of the non-textile polymeric articles include food packages, trays, gloves, blood bags, catheters, ventilation tubes, feeding tubes, transmission tubes, syringes, covers for mobile phones, pipes, toilet seats or toilet seat covers, kitchen sponges, working surface covers, and condoms. In certain embodiments, the material is in a form of a product selected from the group consisting of clothing items, bedding textiles, laboratory or hospital textiles, laboratory equipment, hospital equipment, medical textiles including bandages or sutures and textiles for internal use, personal hygiene articles, packaging or wrapping material, covers for consumer items, food equipment, birth-control devices, agricultural products, or sanitary products.

### 10. Mode of Incorporation

In all embodiments described herein, the antimicrobial polymer compositions may be incorporated by extrusion, injection molding, casting, coating, pouring, additive manufacturing, or combinations thereof. The antimicrobial agents may be embedded within the polymer matrix, coated onto the polymer surface, or both, and may provide antimicrobial activity under light and dark conditions.

In some embodiments, the present invention provides a material for use in combating or inhibiting the activity of microbes or microorganisms, selected from the group consisting of Gram-positive bacteria, Gram-negative bacteria, fungi, parasites, mold, spores, yeasts, protozoa, algae, acarii, and viruses. Each possibility represents a separate embodiment of the invention.

According to some embodiments, the material is for use in combating healthcare-associated infections, nosocomial infections, or a combination thereof. Each possibility represents a separate embodiment of the invention.

Alternatively or additionally, the material of the present invention can be used for the treatment or prevention of atopic fungal, bacterial, and viral infections. The infection may be selected from the group consisting of athlete's foot, yeast infections and staph infections. Each possibility represents a separate embodiment of the invention. In further embodiments, the material is for use in the treatment or prevention of topical viral infections. The infection may be selected from the group consisting of warts and Herpes B. Each possibility represents a separate embodiment of the invention.

### II. Methods of Making

The present disclosure further provides methods for preparing antimicrobial polymer compositions and for manufacturing antimicrobial products and devices comprising such compositions. The methods described herein are particularly advantageous in that the antimicrobial compositions retain efficacy following exposure to elevated temperatures, pressures, and shear forces commonly encountered during polymer processing.

### A. Process for Preparation of the Antimicrobial Polymer

In some embodiments, the antimicrobial polymer composition is prepared by forming a mixture comprising metallic nanoparticles, one or more photosensitizers, and one or more polymers or polymer precursors. In certain embodiments, the metallic nanoparticles and photosensitizer are combined to form a metallic nanoparticle-photosensitizer mixture prior to incorporation into the polymer. The nanoparticles and photosensitizers may be provided in powder, slurry, suspension, or solution form.

In some embodiments, the antimicrobial nanoparticle photosensitizer polymer mixtures are in the form of a solution. Such a solution may be produced by contacting a solution of metallic nanoparticles with a solution of one or more photosensitizers. The mixtures are contacted at any suitable temperature for polymer processing.

In some embodiments, the metallic nanoparticles and photosensitizer are powders. In some embodiments, metallic nanoparticle powder is mixed with one or more powdered photosensitizers and one or more polymers in pellet form. The powders and pellets are contacted at any suitable temperature, for polymer processing.

In some embodiments, the metallic nanoparticles and photosensitizers are dry-mixed prior to addition to the polymer. In other embodiments, the nanoparticles and photosensitizers are first milled, ground, or otherwise processed to improve dispersion, reduce agglomeration, or enhance interaction between components. In some embodiments, the mixture containing the metallic nanoparticle, photosensitizer, and polymer is dry mixed and extruded into molds.

In some embodiments, the antimicrobial compositions are incorporated into one or more polymers to form a composite material. The polymer may be provided as pellets, granules, powders, resins, or liquid precursors, depending on the intended manufacturing method. In certain embodiments, incorporation is achieved by dry blending antimicrobial compositions with polymer pellets or granules, melt blending the antimicrobial compositions into a molten polymer, dispersing the antimicrobial compositions into a polymer solution or resin, forming a masterbatch comprising the antimicrobial compositions and a carrier polymer, followed by dilution with additional polymer material.

In some embodiments, the metallic nanoparticle powder and one or more powdered photosensitizers are mixed with a suitable solution of one or more polymers and ground to make a slurry. In some embodiments, the slurry is then contacted with a second solution containing one or more polymers. The powders and solutions are contacted at any suitable temperature. In some embodiments, the resulting mixture containing the metallic nanoparticle, photosensitizer, and polymer is injected as a liquid into molds. In some embodiments, this results in silicone injection molded material.

The components may be mixed in any proportion to achieve the desired concentration in the mixed solution. In one embodiment, the initial concentrations of each component are selected as required so that the desired concentration in the mixed solution is achieved when equal volumes of metallic nanoparticles, and photosensitizer are mixed together.

In some embodiments, antimicrobial mixture comprises one or more metallic nanoparticles, and one or more photosensitizers. The desired concentration of the nanoparticles in the mixture depends on the desired final concentration in the medical device. This may vary and a suitable choice depends both on the size of the nanoparticle and the concentration of the photosensitizer in the mixture. In some embodiments, the final concentration of the nanoparticles in the mixture is from about 1×10¹¹ to about 5×10¹⁵ particles/ml. In some embodiments, the final concentration is from about 3×10¹¹ to about 1×10¹⁵ particles/ml. In order to obtain such a final concentration, the initial concentration of the nanoparticle solution is from about 1×10¹² to about 1×10¹⁶ particles/ml. If the nanoparticle solution, as prepared or as obtained commercially, is of a higher concentration than this, it may be necessary to dilute the nanoparticle solution before mixing with the photosensitizer. For example, an original nanoparticle solution containing 1×10¹⁴ or 1×10¹⁵ particles/ml may be diluted 1:10 to 1:100, such that the concentration before mixing with the photosensitizer solution is from 1×10¹² to 1×10¹⁴.

In some embodiments, the metallic nanoparticles and the photosensitizer are used in about equal percentages by weight. In some embodiments, the metallic nanoparticles and the photosensitizer are used in different percentages by weight. In some embodiments, the combination of the metallic nanoparticles and the photosensitizer composes about 0.01% to about 10% of the total polymer weight. In some embodiments, the photosensitizer composes about 0.05% to about 20%, 0.05% to 15%, 0.05% to 10%, 0.05% to 5%, 0.05% to 2%, 0.1% to 20%, 0.1% to 15%, 0.1% to 10%, 0.1% to 5%, 0.1% to 2%, 0.5% to 5%, 0.5% to 2%, 1% to 5%, 1% to 2% of the total polymer weight. In some embodiments, the nanoparticles comprise about 0.05% to about 20%, 0.05% to 15%, 0.05% to 10%, 0.05% to 5%, 0.05% to 2%, 0.1% to 20%, 0.1% to 15%, 0.1% to 10%, 0.1% to 5%, 0.1% to 2%, 0.5% to 5%, 0.5% to 2%, 1% to 5%, 1% to 2% of the total polymer weight. In some embodiments, the combination composes about 2% of the total polymer weight. In some embodiments, the combination comprises about 4% of the total polymer weight. In some embodiments, the composition comprises 1% metallic nanoparticles and 1% photosensitizer. In some embodiments, the composition comprises 2% metallic nanoparticles and 2% photosensitizer.

In some embodiments, the initial concentration of the photosensitizer and metallic nanoparticles is chosen such that the final amount of each in the medical device are equal % w/w. In preferred embodiments, the medical device contains 1% w/w ZnO and 1% w/w of MB.

In some embodiments, the amount of polymer used is chosen such that, when extruded, molded, cast, or 3D printed, the volume is sufficient to fill a mold of the desired implantable device.

### B. Manufacture of an Antimicrobial Implant

The methods described herein are for manufacturing an antimicrobial implant intended for use as a medical device. These medical devices tend to be softer polymers, such as drains, catheters, endotracheal tubes, sutures, etc.

The methods may use methods of manufacturing of polymer devices that include some form of melting or shaping, whether through pressure or molding. Examples may include, but are not limited to tubing or catheters through extrusion, housings or syringes through injection molding, containers through blow molding, trays/packaging through thermoforming, fibers/sutures through melt spinning, extrusion and drawing, braiding, nonwovens through melt blowing, and novel or custom devices through additive (FDM/SLS).

In some particular embodiments, the methods provided herein comprise forming the antimicrobial metallic nanoparticle-photosensitizer polymer mixture with the compositions provided herein. In some embodiments, the methods comprise preparing antimicrobial medical devices containing a metallic nanoparticle-photosensitizer mixture by heat- or pressure-based incorporation into a polymer or polymer matrix. In some embodiments, the structure or shape of the medical device is formed at the same time as the metallic nanoparticle-photosensitizer mixture is incorporated. In some embodiments, the method comprises extrusion of the polymer. In some embodiments, the method comprises injection molding. In some embodiments, the method comprises silicone pouring. In some embodiments, the method comprises heating the antimicrobial metallic nanoparticle-photosensitizer polymer mixture to an approximate melt flow temperature of the polymer stock material to produce a molten antimicrobial metallic nanoparticle-photosensitizer polymer mixture. In some embodiments, the method further comprises forming the molten antimicrobial metallic nanoparticle-photosensitizer polymer mixture into the shape of a medical device using 3D printing by fused deposition modeling.

In some embodiments, the methods described herein were surprisingly and unexpectedly found to be preferable to swell encapsulation, a method previously documented in U.S. patent application Ser. No. 19/201,759, for preparing medical devices and implants. In some embodiments, the methods described herein were found to be preferable for preparing medical devices comprising hard polymers and fluorinated polymers such as polytetrafluoroethylene (PTFE), polyvinylidene fluoride (PVDF), and fluorinated ethylene propylene (FEP), in part because the methods described herein solve known issues involving solubility and avoid the use of hazardous fluorinated solvents.

In some embodiments, the methods disclosed herein provide significant advantages over polymer systems photocured during three-dimensional (3D) printing processes as described in WO2024127031A1. Photocured 3D-printed polymers rely on localized light exposure to induce curing, which can result in incomplete polymerization, limited penetration depth, and non-uniform incorporation of light-absorbing additives such as photosensitizers or metallic nanoparticles. In contrast, the present methods employ thermal, pressure-driven, or chemically initiated processing techniques that enable homogeneous bulk dispersion of antimicrobial compositions throughout the polymer matrix without layer-by-layer defects or shadowing effects. These methods further avoid photodegradation of active agents during fabrication and produce mechanically consistent antimicrobial articles that exhibit activity under both light and dark conditions, making them particularly well suited for implantable, enclosed, or load-bearing medical devices.

In some embodiments, the present invention relates to methods of preparing antimicrobial medical devices by blending a mixture of metallic nanoparticles and photosensitizer(s) with a plastic mix prior to shaping the device. The mixture of metallic nanoparticles and photosensitizer(s) provides antibacterial activity in both light and dark conditions. The mixture of metallic nanoparticles and photosensitizer(s) is active at about 2% wt/wt of the total mixture.

Medical devices such as tubing are commonly manufactured by extrusion, including catheter shafts, IV lines, pump tubing, and protective sheaths. Medical devices such as inplantable or insertable devices, surgical tools, and other medical products including syringes, test tubes, and petri dishes are commonly manufactured by injection molding. As described herein, all devices like these may be impregnated with a mixture of metallic nanoparticles and photosensitizer(s) by mixing the mixture of metallic nanoparticles and photosensitizer(s) with plastic pellets or granules used to generate the device, prior to addition to the hopper. In some embodiments, the mixture of metallic nanoparticles and photosensitizer(s) is incorporated into the pellets or granules.

In some embodiments, methylene blue and ZnO antimicrobial compositions are added directly to the polymer prior to it going through the extrusion process. Once the polymer is in pellet form or being melted down, prior to extrusion, MB and ZnO are mixed in with the melted pellet then the polymer is extruded directly with the MB and ZnO mixed in. In some embodiments, the MB and ZnO are directly incorporated into the pellets. This is how many medical devices are manufactured, such as sutures, catheters, and endotracheal tubes, among others. The methods described herein are advantageous over existing swell encapsulation methods, which occur after the manufacturing process. Additionally, the disclosed methods enable uniform incorporation of antimicrobial compositions without reliance on light exposure, overcoming curing depth and shadowing limitations associated with photocured polymers.

### C. Extrusion of an Antimicrobial Polymer

In one aspect, the antimicrobial metallic nanoparticle-photosensitizer polymers and medical devices thereof are manufactured by extrusion. In some embodiments, a method for extrusion comprises mixing the polymer with the metallic nanoparticle and photosensitizer for embedding the metallic nanoparticle and photosensitizer in the polymer; extruding, coextruding, laying down, or forming the antimicrobial metallic nanoparticle-photosensitizer polymer.

In a preferred embodiment, a method of manufacturing an antimicrobial metallic nanoparticle-photosensitizer composition comprises the steps of: mixing a polymer with a metallic nanoparticle and photosensitizer for embedding the metallic nanoparticle and photosensitizer uniformly throughout the polymer; extruding, forming or laying down the polymer in a single or reactively bonded multilayer film.

Plastic extrusion is a process involving melting of plastic and reformation into a continuous profile. In extrusion, materials are typically obtained in the shape of pellets or granules, then the pellets and granules are fed through a hopper into the barrel of an extruder. Inside the barrel of the extruder, a screw typically rotates to move the pellets through the extruder. As they are moved through the unit, heating bands around the barrel warm the pellets or granules, slowly warming them, resulting in molten plastic by the end of the barrel. The material is then forced into a die, which shapes the polymer or plastic into a desired shape, which hardens during cooling.

Medical devices are commonly manufactured by extrusion methods, including catheters and other tubing (see WO2000001420A2, U.S. Pat. No. 8,431,060).

In some embodiments, a multilayered film is fabricated by methods involving extrusion, loss of solvent, reactive forming, pressure forming, and other known methods of forming a layered or non-layered three-dimensional structure, which may have a uniform or non-uniform cross-section.

In some embodiments, the metallic nanoparticle-photosensitizers are incorporated into the polymer through a master batch manufacturing process. Thus, according to some embodiments, the intermediate product is a master-batch.

In another embodiment, the present invention provides a method for the preparation of a material having antimicrobial properties, said material comprising a polymer having incorporated therein the antimicrobial compositions provided herein, the antimicrobial compositions being incorporated substantially uniformly within said polymer, the method comprising the steps of: a) processing the antimicrobial compositions; and b) mixing said antimicrobial compositions with at least one polymer.

According to some embodiments, step a) comprises processing the antimicrobial compositions to obtain particles having mean particle sizes which are inversely proportional to the specific gravity thereof. In some embodiments, said processing comprises grinding.

In some embodiments, the extrusion occurs at a high temperature. In some embodiments, the extrusion occurs between 15° and 350° C., 150 and 300° C., 150 and 250° C., 150 and 200° C., 150 and 190° C., 160 and 190° C., 160 and 180° C. The antimicrobial compositions of photosensitizer and nanoparticles were surprisingly found to survive the high temperatures of extrusion, still exhibiting antimicrobial properties in both dark and light conditions.

In some embodiments, the method further comprises a step of encapsulating the metal oxide powder particles within an encapsulating compound. In other embodiments, the method comprises a step of mixing the metal oxide powders with a metal deactivating agent or a chelating agent. In further embodiments, the method comprises a step of mixing the antimicrobial compositions with a surfactant.

According to further embodiments, the method comprises producing a master batch, comprising the metallic nanoparticle-photosensitizer mixture compositions and a polymer. According to some embodiments, the master batch is homogeneous. The master batch may be formed into pellets. Alternatively, the master batch may be formed into granules.

In some embodiments, step b) further comprises adding the master batch to a polymer slurry. In further embodiments the polymer slurry comprises a polymer, which is the same as the carrier polymer. In other embodiments, the polymer slurry comprises a polymer, which is chemically compatible with the carrier polymer.

According to some embodiments, the method further comprises step c) comprising forming from the obtained mixture a film, a foil, a fiber, a yarn, a fiber, a textile, a textile product or a non-textile polymeric article. Each possibility represents a separate embodiment of the invention.

According to some particular embodiments, the method further comprises step c) comprising forming from the obtained mixture a film, a foil, a fiber, a yarn, a fiber or a textile, comprising said antimicrobial compositions. Each possibility represents a separate embodiment of the invention. According to other particular embodiments, the method further comprises step c), comprising forming from the obtained mixture a textile product or a non-textile polymeric article, comprising said antimicrobial compositions. Each possibility represents a separate embodiment of the invention. In some embodiments, the textile product or non-textile polymeric article is a medical device.

In some embodiments, step c) comprises extrusion, molding, casting, or 3D printing. In some exemplary embodiments step c) comprises extrusion. In further embodiments, extrusion comprises spinning through a spinneret. In the preferred embodiments, the material is homogeneously extruded. In other embodiments step c) comprises molding.

In some embodiments, step c) comprises forming a polymeric fiber from the mixture obtained in step b. In some embodiments, the method further comprises blending the polymer fiber with a natural fiber. In some embodiments, the method comprises forming the fiber into yarn, textile, fabric or a textile product.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### D. Injection Molding and Silicone Pouring

In an aspect, the present invention relates to methods for preparing the antimicrobial metallic nanoparticle-photosensitizer polymers and medical devices thereof by injection molding.

Injection molding is similar to extrusion but differs in that the materials are produced by injecting molten material into a mold. Traditional injection molding injection steps occur at high pressure, but the process may also be performed at low temperature for parts which are sensitive or require less stress. Injection molding is most commonly used with thermoplastic and thermoset polymers but can be performed with a variety of other materials, as detailed elsewhere. Molten material is injected into the cavity of the mold, where it cools and hardens into the shape of the cavity. Injection molding typically uses a special injection molding machine comprising an injection unit, a mold, and a clamp. Injection molding is the most common method currently of manufacturing plastic products.

In injection molding, materials are typically obtained in the shape of pellets or granules, then the pellets and granules are fed through a hopper into the barrel of an injection unit. Inside the barrel, a screw typically rotates to move the pellets through the injection unit. As they are moved through the unit, heating bands around the barrel warm the pellets or granules, slowly warming them, resulting in molten plastic by the end of the barrel. Once enough molten plastic is collected at the end of the barrel, the screw moves forward, pushing the molten plastic into the mold in an injection-type step. The plastic is then allowed to solidify, the mold opens, and the solidified plastic device is ejected. The process can then repeat.

Injection molding is one of the primary manufacturing methods for medical devices. Devices including suture anchors, bone plates and screws, endoscope housings, implantable device housings, leads for pacemakers and defibrillators, and implantable dental components, among others, are commonly manufactured by injection molding. Medical devices produced by injection molding are tough, durable, often rigid and chemically resistant, and often retain their properties after repeated sterilization. Medical devices are commonly manufactured by injection molding methods (see US Publication No. US20050033237A1, U.S. Pat. No. 11,981,757). Types of medical devices that may be produced by injection molding as described herein include but are not limited to respiratory devices, syringe parts, catheters, cannulas, hearing aid tips and buds, diaphragms, gaskets, pump housings, valves, seals, washers, and other components.

In another aspect, the present invention relates to methods for preparing the antimicrobial metallic nanoparticle-photosensitizer polymers and medical devices thereof by silicone pouring. Silicone pouring, or silicone casting is a process involving pouring a casting material, such as a silicone, into a silicone mold. The molds described in this disclosure may be in any shape or configuration. One of skill in the art would understand that the mold may be selected based on the type of material and the intended use of the product. One of skill in the art could understand that it would be desirable to make a variety of different shapes and devices exhibiting antimicrobial or antibacterial properties, especially wherein the antimicrobial or antibacterial properties persist under dark conditions.

There are no special restrictions on the materials which may be used with the present invention. In some embodiments, the materials are polymers. In some embodiments, the materials are polyolefins, polystyrene, ABS resins, AS resins, PVC resins, methacrylic resins and fluorine based resins, saturated polyester resins, polycarbonate resins, polyarylate resins, polyacetal resins, polysulfones, and modified polyphenylene ether resins. In particular embodiments, they are silicone polymers. In some embodiments, the materials are liquid silicone rubber. In some embodiments, the materials are a combination of silicone and thermoplastic materials, and the process involves two-shot silicone-thermoplastic molding.

The inventors of the present invention have found that introducing the metallic nanoparticle/photosensitizer mixture to injection molded components, and in particular medical devices, provides antimicrobial medical devices made of a multitude of materials. In some embodiments, as described herein, these materials and devices exhibit antibacterial activity, and in some embodiments, said antibacterial activity persists in dark conditions.

In some embodiments, a method of producing antimicrobial compositions by liquid injection or silicone pouring comprises a) combining a photosensitizer with metallic nanoparticles and a resin solution to generate a slurry; and b) pouring or injecting into the desired container. In some embodiments, the photosensitizer and metallic nanoparticles are ground into the resin solution. In some embodiments, the process further comprises mixing the slurry with a second solution. In some embodiments, the second solution contains an active material to initiate polymerization. In some embodiments, the method further comprises a step of allowing the resulting poured or injected mixture to cure in the desired container.

In some embodiments, the resin solution comprises precipitated silica, silicone dioxide, and tetrapropyl orthosilicate. In some embodiments, the resin solution comprises vinyl silicone oil, silicon dioxide and tetrapropyl orthosilicate. In some embodiments, these ingredients are optionally in 62% w/w, 37% w/w, and 1% w/w, respectively. In some embodiments, the second solution comprises precipitated silica, silicon dioxide, and Me hydrogen. In some embodiments, the second solution comprises vinyl silicone oil, silicone dioxide, and poly(methylhydrosiloxane), optionally in 61% w/w, 37% w/w, and 2% w/w.

A mixture of precipitated silica (6.2 g), silicone dioxide (3.7 g), and tetrapropyl orthosilicate (0.1 g) was placed in a suitable container. To this was added Rose Bengal lactone (RBL) (192 mg) and 50 nm zinc oxide (768 mg). This mixture was then stirred until the RBL and ZnO were fully dispersed within the matrix. Next, a mixture of precipitated silica (6.1 g), silicone dioxide (3.7 g), and Me hydrogen (0.2 g) were added, and the resulting viscous liquid was stirred until fully homogenous. The final material was allowed to cure overnight.

In some embodiments, the grinding step is performed for between 5 and 30 minutes. In some embodiments, the grinding step is performed for at least 10 minutes.

### III. Methods of Treatment

### A. Antimicrobial Effect of the Mixtures

The mixtures of the present invention have an antimicrobial effect, i.e. they are capable of killing or inhibiting the growth of microorganisms, including bacteria, viruses, fungi and prions, that can cause disease in humans, animals or plants. In some embodiments, the mixtures of the present invention are used to kill or inhibit the growth of Staphylococcus aureus. Staphylococcus aureus as used in this application shall also include Methicillin-Resistant Staphylococcus aureus (“MRSA”). The mixtures of the present invention may also be used to kill or inhibit the growth of Propionibacterium acnes, Escherichia coli, and Candida albicans.

In other embodiments, the mixtures of the present invention are used to kill or prevent the growth of the microbes involved in in wound infections and in disinfecting or sterilizing wounds and other lesions on a subject. Thus, the mixtures of the present invention may be used to kill or inhibit the growth of Streptococcus sanguis, Porphyromonas gingivalis, Fusobacterium nulceatum, Actinobacillus actinomycetemcomitans, Candida albicans, Streptococcus mutans and lactobacilli, Acetobacter aurantius, Acinetobacter baumannii, Actinomyces israelii, Agrobacterium radiobacter, Agrobacterium tumefaciens, Anaplasma phagocytophilum, Azorhizobium caulinodans, Azotobacter vinelandii, viridans streptococci, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus thuringiensis, Bacteroides fragilis Bacteroides gingivalis, Bacteroides melaninogenicus (also known as Prevotella melaninogenica), Bartonella henselae, Bartonella quintana, Bordetella bronchiseptica Bordetella pertussis, Borrelia burgdorferi, Brucella abortus, Brucella melitensis, Brucella suis, Burkholderia, Burkholderia mallei, Burkholderia pseudomallei, Burkholderia cepacian, Calymmatobacterium granulomatis, Campylobacter coli, Campylobacter fetus, Campylobacter jejuni, Helicobacter pylori, Chlamydia trachomatis, Chlamydophila, Chlamydophila pneumoniae (also called Chlamydia pneumoniae), Chlamydophila psittaci (also called Chlamydia psittaci), Clostridium botulinum, Clostridium difficile, Clostridium perfringens (also called Clostridium welchii), Clostridium tetani, Corynebacterium, Corynebacterium diphtheriae, Corynebacterium fusiforme, Coxiella burnetiid, Ehrlichia chaffeensis, Ehrlichia ewingii, Eikenella corrodens, Enterobacter 17 5 10 15 20 25 30 35 cloacae, Enterococcus avium, Enterococcus durans, Enterococcus faecalis, Enterococcus faecium, Enterococcus gallinarum, Enterococcus maloratus, Escherichia coli, Fusarium, Fusobacterium necrophoru, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus bulgaricus, Lactobacillus casei, Lactococcus lactis, Legionella pneumophila, Leptospira interrogans, Leptospira noguchii, Listeria monocytogenes, Methanobacterium extroquens, Microbacterium multiforme, Micrococcus luteus, Moraxella catarrhalis Mycobacterium, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Mycoplasma Mexican, Neisseria gonorrhoeae, Neisseria meningitidis, Pasteurella multocida, Pasteurella tularensis, Peptostreptococcus, Porphyromonas gingivalis, Prevotella melaninogenica (previously called Bacteroides melaninogenicus), Pseudomonas aeruginosa, Rhizobium radiobacter, Rickettsia prowazekii, Rickettsia psittaci, Rickettsia quintana, Rickettsia rickettsia, Rickettsia trachomae, Rochalimaea, Rochalimaea henselae, Rochalimaea quintana, Rothia dentocariosa, Salmonella enteritidis, Salmonella typhi, Salmonella typhimurium, Serratia marcescens, Shigella dysenteriae, Spirillum volutans, Staphylococcus aureus, Staphylococcus epidermidis, Stenotrophomonas maltophilia Streptococcus agalactiae, Streptococcus avium, Streptococcus bovis, Streptococcus cricetus, Enterococcus faecium, Enterococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema, Ureaplasma urealyticum, Vibrio cholerae, Vibrio comma, Vibrio parahaemolyticus, Vibrio vulnificus, Wolbachia, Yersinia enterocolitica, Yersinia pestis, Yersinia pseudotuberculosis. The bacteria may be selected from Acinetobacter baumannii, Pseudomonas aeruginosa, Enterobacteriaceae, Enterococcus faecium, Staphylococcus aureus, Helicobacter pylori, Campylobacter spp, Salmonellae, Neisseria gonorrhoeae, Haemophilus influenzae, Shigella spp. The bacteria may be a bacteria resistant to one or more antibiotics, for example a bacterium selected from Acinetobacter baumannii (carbapenem-resistant), 18 Pseudomonas aeruginosa (carbapenem-resistant), Enterobacteriaceae (carbapenem resistant, ESBL-producing), 5 Enterococcus faecium (vancomycin-resistant), Staphylococcus aureus (methicillin-resistant and/or vancomycin-intermediate and resistant), Helicobacter pylori (clarithromycin-resistant), Campylobacter spp., (fluoroquinolone-resistant), Salmonellae (fluoroquinolone resistant), Neisseria gonorrhoeae (cephalosporin-resistant and/or fluoroquinolone resistant), Streptococcus pneumoniae (penicillin-non-susceptible), Haemophilus influenzae (ampicillin-resistant), Shigella spp., (fluoroquinolone-resistant). The microorganism may also be a fungus, including but not limited to species of Candida, Cryptococcus, Aspergillus, Fusarium, Trichophyton, Microsporum, Epidermophyton, Mucor, and Rhizopus.

In some embodiments, the compositions of photosensitizers and nanoparticles disclosed herein may exhibit synergistic antibacterial effects.

### B. Applications of Mixtures

The invention provides the use of the compositions of the present invention in the manufacture of a medical device. These medical devices may be used to reduce microbial activity on medical equipment and textiles, of tissues of a body cavity, or at the site of a wound or lesion.

The wound or lesion treated may be any surgical or trauma-induced wound, a lesion caused by a disease-related microbe, or a wound or lesion infected with such a microbe. The medical device may be chosen to disinfect or sterilize a wound or lesion as a routine precaution against infection or as a specific treatment of an already diagnosed infection of a wound or lesion. In some embodiments, the body cavity is the oral cavity. The present invention may also be used in other body cavities, such as the nose, rectum, vagina, etc.

In another aspect, the invention provides the use of a mixture of the present invention in the manufacture of an implantable medical device for use in killing or preventing the growth of disease-related microbes in any body orifices or any body cavity such as the oral cavity, nose, rectum, vagina, ear, eyes, inside any incision/wound/cut etc., or on implanted devices by contacting the microbes.

The medical device may be left in contact with the microbes for a period of time. This duration of time may vary depending on the particular photosensitizer in use and the target microbes to be killed. Time of contact will entirely depend on the best course of action for the device (e.g., hours, days, to weeks). The mixture will start being antimicrobial instantly and has been shown to have a 2-log reduction >99.99% in activity against a microbe. In some embodiments, the duration of time of contact can be from about 1 second to about 10 minutes. In some embodiments, the duration of time is about 10 minutes to about 1 hour. In other embodiments, the duration of time is about 1 hour to about 3 hours. In yet other embodiments, the duration of time is about 3 hours to about 5 hours. In other embodiments, the antimicrobial mixture is left in the body cavity indefinitely.

### EXAMPLES

### Example 1: Bacterial Testing of Samples

### Materials and Methods

### Day 0:

A single bacterial pellet was placed into a 15 mL sterile tube. A milliliter of Dulbecco's phosphate buffered saline (DPBS) was added to the pellet, and the pellet was allowed to fully dissolve over the course of 5 minutes. The solution was the vortexed for 10 seconds. A 100 μL aliquot was removed from the solution and placed on an agar plate. A spreader was use to spread an even layer of the solution around the plate. An agar plate box was sterilized by washing with 70% isopropyl alcohol (IPA), and a wetted blue roll was placed at the bottom of the box. The agar plate was placed into the box on a raised plastic platform and incubated.

### Day 1:

After incubation, individual colonies were collected, streak plated and incubated.

### Day 2:

A 10 mL aliquot of Nutrient broth was added to a centrifuge tube. A single colony from the plate prepared on Day 1 was collected and mixed into the nutrient broth, and the tube was vortexed for 10 seconds. The prepared tube was placed in an incubator at 250 rpm at a temperature of 37° C. overnight.

### Day 3:

The centrifuge tube from Day 2 was centrifuged at 3800 rpm for 10 minutes. The supernatant was removed, and 10 mL of DPBS was added. This mixture was vortexed for 10 seconds. This was repeated once.

Optionally, the bacterium was resuspended into 1 mL DPBS. A 100 μL aliquot was added to a container containing 1.9 mL DPBS. This was vortexed to mix. The absorbance of this solution was measured at 600 nm, and Table 1 was used to calculate an amount of DPBS needed to dilute the stock bacterium. 200 μL of stocks were measured into new containers, and the calculated volume of DPBS was added to each.

TABLE 1

| | | Volume of |
|---|---|---|
| Absorbance | | DPBS to add to |
| 600 nm | Stock CFU/mL | stock (mL) |
| 0.667 | 7.79E+09 | 3.117 |
| 0.675 | 7.90E+09 | 3.160 |
| 0.661 | 7.71E+09 | 3.084 |

In instances where the absorbance was not measured, the stock was not diluted. In all instances, a 100 μL aliquot of the inoculum was added to a first well of a well plate. The inoculum was serially diluted ten times into wells each containing 120 μL PBS by taking 30 μL aliquots from the previous well. In duplicate, 5 μL of each was plated in a line on nutrient agar.

The desired sample was plated on a glass slide, and 25 μL of inoculum was added to the sample. This was placed in a humidity chamber overnight.

The sample bacterium tested against was selected from Escherichia coli (ATCC 8739), Candida albicans (ATCC 10231), and Staphylococcus epidermidis (ATCC 12228), as indicated in the Examples. A 25 μL inoculum was added to each sample, and spot plates were prepared with 5 μL at each dilution level.

### Example 2: Preparation of Extruded Materials

Extruded plastic materials were generated containing methylene blue and zinc oxide. To generate these, samples were heated to relevant extrusion temperature and mixed to imitate the extrusion process.

Polycaprolactone (PCL) (9 g) was added to an aluminum crucible. The crucible was heated to 140° C. and the molten PCL was mixed at 30 rpm. Methylene blue (MP), 2% by weight, and zinc oxide (ZnO), 8% by weight (<50 nm particle size), were added to the crucible. The resulting material was mixed for 20 minutes. Once mixing was completed, the resulting PCL mixture was cut to shape and allowed to cool in air.

Various compositions of extruded materials were tested for antibacterial properties, as outlined in the following Examples, and with the procedure outlined in Example 1. In these Examples, the best-performing compositions were determined. For Gram positive bacteria (Staphylococcus epidermidis), the best compositions were found to be methylene blue with zinc oxide and crystal violet with zinc oxide. For Gram negative bacteria (Escherichia coli), the best composition was found to be methylene blue with zinc oxide and crystal violet with zinc oxide. For fungi (Candida albicans), the best composition was found to be crystal violet with zinc oxide.

Further, experiments were run with only methylene blue (without metallic nanoparticles) and with ZnO (without any photosensitizer), testing against E. coli, according to the procedure above. With 2 w/w % methylene blue and no metallic nanoparticles, the PCL sample exhibited 99.99% mean % log reduction in light conditions and 72.91 mean % log reduction in dark conditions. With 2 w/w % ZnO and no photosensitizer, the PCL sample exhibited 0% mean % log reduction in light conditions and 78.29% mean % log reduction in dark conditions. A PCL sample with 2 w/w % ZnO and 2 w/w % methylene blue provided 99.99% mean % log reduction in light conditions and 96.80% mean % log reduction in dark conditions.

### Example 3: Variation of Photosensitizer in Extruded Materials

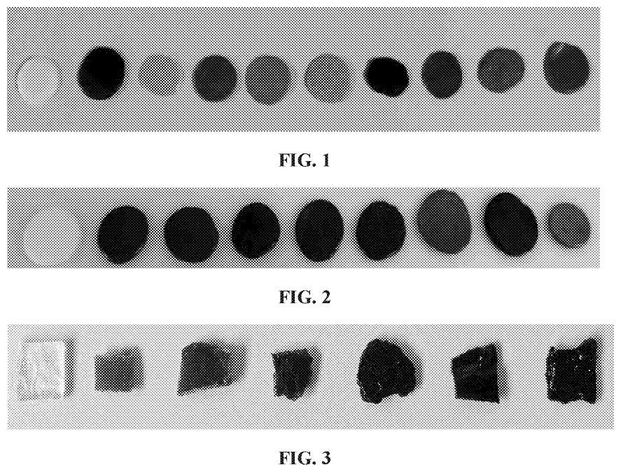

Using the process as described in Example 2, the photosensitizer (methylene blue) was exchanged with the following compounds at the same percentage weight (2%): 1) Tris(2,2′-bipyridine)ruthenium(II) hexafluorophosphate; 2) Quercetin hydrate; 3) Curcumin; 4) Zn(II) Phthalocyanine; 5) Protoporphyrin IX; 6) Crystal Violet; 7) Azure-A; 8) Eosin-Y disodium salt. The products of this process are represented in FIG. 1, showing PCL with MB replacements. From left to right, FIG. 1 shows: PCL (without any methylene blue, ZnO, or replacements), MB, Tris(2,2′-bipyridine)ruthenium(II) hexafluorophosphate, Quercetin hydrate, Curcumin, Zn(II) Phthalocyanine, Protoporphyrin, Crystal Violet, Azure-A, and Eosin Y disodium salt.

Antibacterial testing was conducted with all materials described above. Antibacterial testing was carried out against Escherichia coli (ATCC 8739), Candida albicans (ATCC 10231), and Staphylococcus epidermidis (ATCC 12228). Each test was carried out in biological duplicate.

Samples tested for performance in the light were left on a table top within a room lit by LED lights at Ca. 250-350 Lux. Samples tested in the dark were left in the absence of light by placing them into a closed cabinet.

All test samples were left at room temperature for ~18 hours. Once complete, the solution was resuspended and serial dilutions were spot-plated to determine bacterial numbers.

### Results

Each of Escherichia coli, Candida albicans, and Staphylococcus epidermidis were tested against the compositions described above in both light and dark conditions. Tables 2 and 3 describe testing against E. coli. Tables 4 and 5 describe testing against C. albicans. Tables 6 and 7 describe testing against S. epidermidis. In these results, the inoculum was a bacteria count without exposure to any sample. The control was a bacteria count after exposure to a control sample (i.e. a plain polymer with no methylene blue, zinc oxide, or replacements thereof).

### Methylene Blue Substitutes

TABLE 2
Testing against E. coli in light conditions.

| | | | Standard |
|---|---|---|---|
| | Mean % Log | Mean | deviation |
| Description | reduction | CFU/mL | CFU/mL |
| Inoculum | n/a | 3.91E+08 | 1.55E+07 |
| Control | n/a | 4.70E+07 | 1.03E+07 |
| Tris(2,2′- | 46.97% | 2.75E+07 | 1.73E+07 |
| bipyridine)ruthenium(II) | | | |
| hexaflurophosphate | | | |
| Quercetin hydrate | 27.22% | 4.39E+07 | 3.97E+07 |
| Curcumin | 99.97% | 1.88E+04 | 1.66E+04 |
| Zinc phthalocyanine | 0.00% | 5.59E+07 | 8.32E+06 |
| Protoporphyrin IX | 10.15% | 4.21E+07 | 5.15E+06 |
| Crystal violet | >99.99% (limit | <2.00E+02 | 0 |
| | of detection) | | |
| Azure A chloride | >99.99% (limit | <2.00E+02 | 0 |
| | of detection) | | |
| Eosin Y disodium salt | >99.99% (limit | <2.00E+02 | 0 |
| | of detection) | | |
| Methylene Blue | >99.99% (limit | <2.00E+02 | 0 |
| | of detection) | | |

TABLE 3
Testing against E. coli in dark conditions.

| | | | Standard |
|---|---|---|---|
| | Mean % Log | Mean | deviation |
| Description | reduction | CFU/mL | CFU/mL |
| Inoculum | n/a | 3.91E+08 | 1.55E+07 |
| Control | n/a | 3.10E+08 | 4.16E+07 |
| Tris(2,2′- | 30.74% | 2.13E+08 | 2.08E+07 |
| bipyridine)ruthenium(II) | | | |
| hexaflurophosphate | | | |
| Quercetin hydrate | 57.88% | 1.56E+08 | 1.22E+08 |
| Curcumin | 29.45% | 2.17E+08 | 5.31E+06 |
| Zinc phthalocyanine | 11.69% | 2.73E+08 | 2.16E+07 |
| Protoporphyrin IX | 54.61% | 1.70E+08 | 1.36E+08 |
| Crystal violet | 82.07% | 5.52E+07 | 3.15E+06 |
| Azure A chloride | 27.14% | 2.24E+08 | 1.56E+07 |
| Eosin Y disodium salt | 62.68% | 1.20E+08 | 4.86E+07 |
| Methylene Blue | 88.91% | 4.82E+07 | 4.82E+07 |

TABLE 4
Testing against C. albicans in light conditions.

| | | | Standard |
|---|---|---|---|
| | Mean % Log | Mean | deviation |
| Description | reduction | CFU/mL | CFU/mL |
| Inoculum | n/a | 6.02E+08 | 3.90E+07 |
| Control | n/a | 8.25E+07 | 4.16E+06 |
| Tris(2,2′- | 1.71% | 8.08E+07 | 2.00E+06 |
| bipyridine)ruthenium(II) | | | |
| hexaflurophosphate | | | |
| Quercetin hydrate | 81.42% | 1.53E+07 | 8.32E+05 |
| Curcumin | 81.42% | 1.53E+07 | 4.16E+05 |
| Zinc phthalocyanine | 68.67% | 4.00E+07 | 4.32E+07 |
| Protoporphyrin IX | 44.47% | 6.27E+07 | 6.09E+07 |
| Crystal violet | 99.99% (limit | <2.00E+02 | 0 |
| | of detection) | | |
| Azure A chloride | 37.05% | 5.38E+07 | 1.98E+07 |
| Eosin Y disodium salt | 94.21% | 5.92E+06 | 4.94E+06 |
| Methylene Blue | 96.56% | 6.49E+06 | 8.27E+06 |

TABLE 5
Testing against C. albicans in dark conditions.

| | | | Standard |
|---|---|---|---|
| | Mean % Log | Mean | deviation |
| Description | reduction | CFU/mL | CFU/mL |
| Inoculum | n/a | 6.02E+08 | 3.90E+07 |
| Control | n/a | 1.20E+08 | 1.55E+07 |
| Tris(2,2′- | 3.40% | 1.15E+08 | 7.31E+06 |
| bipyridine)ruthenium(II) | | | |
| hexaflurophosphate | | | |
| Quercetin hydrate | 49.18% | 7.14E+07 | 5.31E+07 |
| Curcumin | 24.75% | 9.33E+07 | 3.62E+07 |
| Zinc phthalocyanine | 0.00% | 1.35E+08 | 3.13E+07 |
| Protoporphyrin IX | 27.64% | 8.67E+07 | 1.25E+07 |
| Crystal violet | 99.99% | 2.59E+04 | 2.98E+04 |
| Azure A chloride | 0.00% | 3.48E+08 | 3.29E+08 |
| Eosin Y disodium salt | 0.00% | 1.77E+08 | 9.21E+07 |
| Methylene Blue | 0.00% | 2.82E+08 | 2.56E+08 |

TABLE 6
Testing against S. epidermidis in light conditions.

| | | | Standard |
|---|---|---|---|
| | Mean % Log | Mean | deviation |
| Description | reduction | CFU/mL | CFU/mL |
| Inoculum | n/a | 7.81E+06 | 4.08E+06 |
| Control | n/a | 5.11E+06 | 3.15E+06 |
| Crystal Violet/ZnO | >99.99% (limit of | <2.00E+02 | 0 |
| | detection) | | |
| Eosin-Y/ZnO | >99.99% (limit of | <2.00E+02 | 0 |
| | detection) | | |
| MB/ZnO | >99.99% (limit of | <2.00E+02 | 0 |
| | detection) | | |

TABLE 7
Testing against S. epidermidis in dark conditions.

| | Mean % Log | Mean | Standard |
|---|---|---|---|
| Description | reduction | CFU/mL | deviation CFU/mL |
| Inoculum | n/a | 7.81E+06 | 4.08E+06 |
| Control | n/a | 9.83E+06 | 3.92E+06 |
| Crystal Violet/ZnO | 99.92% | 1.41E+05 | 1.99E+05 |
| Eosin-Y/ZnO | 96.03% | 3.76E+05 | 4.95E+04 |
| MB/ZnO | 92.53% | 7.65E+05 | 4.17E+05 |

### Example 4: Variation of Metallic Nanoparticle in Extruded Materials

Using the process as described in Example 2, the photosensitizer (methylene blue) was exchanged with the following compounds at the same percentage weight (8%): 1) silver nanoparticles (<100 nm); 2) copper nanoparticles (40-60 nm); 3) cupric oxide nanoparticles (<50 nm); 4) magnesium oxide nanoparticles (50 nm); 5) tungsten (VI) oxide nanoparticles (<100 nm); 6) iron (II,III) oxide nanoparticles (50-100 nm); 7) graphene oxide powder (average number of layers 15-20); and 8) titanium dioxide nanoparticles (<100 nm). The products of this process are represented in FIG. 2, showing PCL with ZnO replacements. From left to right, FIG. 2 shows: PCL (without any methylene blue, ZnO, or replacements), ZnO, silver nanoparticles, copper nanoparticles, cupric oxide nanoparticles, magnesium oxide nanoparticles, tungsten (VI) oxide nanoparticles, iron (II,III) oxide nanoparticles, and titanium dioxide nanoparticles.

Antibacterial testing was conducted with all materials described above. Antibacterial testing was carried out against Escherichia coli (ATCC 8739), Candida albicans (ATCC 10231), and Staphylococcus epidermidis (ATCC 12228). Each test was carried out in biological duplicate.

Samples tested for performance in the light were left on a table top within a room lit by LED lights at Ca. 250-350 Lux. Samples tested in the dark were left in the absence of light by placing them into a closed cabinet.

All test samples were left at room temperature for ~18 hours. Once complete, the solution was resuspended and serial dilutions were spot-plated to determine bacterial numbers.

### Results

Each of Escherichia coli, Candida albicans, and Staphylococcus epidermidis were tested against the compositions described above in both light and dark conditions. Tables 8 and 9 describe testing against E. coli. Tables 10 and 11 describe testing against C. albicans. Tables 12 and 13 describe testing against S. epidermidis.

TABLE 8
Testing against E. coli in light conditions.

| | | | Standard |
|---|---|---|---|
| | Mean % Log | Mean | deviation |
| Description | reduction | CFU/mL | CFU/mL |
| Inoculum | n/a | 6.73E+08 | 3.90E+07 |
| Control | n/a | 5.64E+07 | 3.08E+06 |
| Silver (<100 nm) | >99.99% (limit | <2.00E+02 | 0 |
| | of detection) | | |
| Copper (40-60 nm) | >99.99% (limit | <2.00E+02 | 0 |
| | of detection) | | |
| Copper (II) Oxide | >99.99% (limit | <2.00E+02 | 0 |
| (<50 nm) | of detection) | | |
| Magnesium Oxide | >99.99% (limit | <2.00E+02 | 0 |
| (<50 nm) | of detection) | | |
| Tungsten (VI) Oxide | 99.98% | 4.94E+04 | 2.59E+04 |
| (<100 nm) | | | |
| Iron (II, III) Oxide | >99.99% (limit | <2.00E+02 | 0 |
| (50-100 nm) | of detection) | | |
| Graphene Oxide | 99.98% | 5.88E+04 | 3.13E+04 |
| Titanium Dioxide | >99.99% (limit | <2.00E+02 | 0 |
| | of detection) | | |
| Zinc Oxide (50 nm) | >99.99% (limit | <2.00E+02 | 0 |
| | of detection) | | |

TABLE 9
Testing against E. coli in dark conditions.

| | | | Standard |
|---|---|---|---|
| | Mean % Log | Mean | deviation |
| Description | reduction | CFU/mL | CFU/mL |
| Inoculum | n/a | 6.73E+08 | 3.90E+07 |
| Control | n/a | 5.05E+07 | 3.67E+06 |
| Silver (<100 nm) | >99.99% (limit | <2.00E+02 | 0 |
| | of detection) | | |
| Copper (40-60 nm) | >99.99% (limit | <2.00E+02 | 0 |
| | of detection) | | |
| Copper (II) Oxide | >99.99% (limit | <2.00E+02 | 0 |
| (<50 nm) | of detection) | | |
| Magnesium Oxide | 95.03% | 2.42E+06 | 1.74E+05 |
| (<50 nm) | | | |
| Tungsten (VI) Oxide | 0% | 1.55E+08 | 1.34E+08 |
| (<100 nm) | | | |
| Iron (II, III) Oxide | 0% | 2.00E+08 | 2.59E+08 |
| (50-100 nm) | | | |
| Graphene Oxide | 0% | 6.32E+07 | 1.05E+07 |
| Titanium Dioxide | 0% | 8.37E+07 | 1.76E+07 |
| Zinc Oxide (50 nm) | 99.54% | 2.75E+05 | 2.26E+05 |

TABLE 10
Testing against C. albicans in light conditions.

| | Mean % | | Standard |
|---|---|---|---|
| | Log | Mean | deviation |
| Description | reduction | CFU/mL | CFU/mL |
| Inoculum | n/a | 6.02E+08 | 3.90E+07 |
| Control | n/a | 1.04E+08 | 6.84E+07 |
| Silver (<100 nm) | 81.14% | 2.37E+07 | 2.27E+07 |
| Copper (40-60 nm) | 96.79% | 8.08E+06 | 1.06E+07 |
| Copper (II) Oxide | 99.86% | 1.29E+05 | 1.66E+04 |
| (<50 nm) | | | |
| Magnesium Oxide | 94.04% | 5.90E+06 | 2.91E+06 |
| (<50 nm) | | | |
| Tungsten (VI) Oxide | 90.55% | 1.02E+07 | 7.26E+06 |
| (<100 nm) | | | |
| Iron (II, III) Oxide | 95.81% | 3.88E+06 | 3.31E+05 |
| (50-100 nm) | | | |
| Titanium Dioxide | 98.66% | 1.68E+06 | 1.61E+06 |
| Zinc Oxide (50 nm) | 93.87% | 6.32E+06 | 3.95E+06 |

TABLE 11
Testing against C. albicans in dark conditions.

| | Mean % | | Standard |
|---|---|---|---|
| | Log | Mean | deviation |
| Description | reduction | CFU/mL | CFU/mL |
| Inoculum | n/a | 6.02E+08 | 3.90E+07 |
| Control | n/a | 1.58E+08 | 2.40E+07 |
| Silver (<100 nm) | 27.05% | 1.15E+08 | 2.16E+06 |
| Copper (40-60 nm) | 39.40% | 9.85E+07 | 3.53E+07 |
| Copper (II) Oxide | 59.31% | 6.70E+07 | 2.82E+07 |
| (<50 nm) | | | |
| Magnesium Oxide | 42.85% | 9.05E+07 | 1.55E+07 |
| (<50 nm) | | | |
| Tungsten (VI) Oxide | 28.47% | 1.13E+08 | 1.88E+07 |
| (<100 nm) | | | |
| Iron (II, III) Oxide | 19.83% | 1.28E+08 | 3.13E+07 |
| (50-100 nm) | | | |
| Titanium Dioxide | 20.75% | 1.25E+08 | 1.45E+07 |
| Zinc Oxide (50 nm) | 30.74% | 1.09E+08 | 0 |

TABLE 12
Testing against S. epidermidis in light conditions.

| | | | Standard |
|---|---|---|---|
| | Mean % Log | Mean | deviation |
| Description | reduction | CFU/mL | CFU/mL |
| Inoculum | n/a | 7.81E+06 | 4.08E+06 |
| Control | n/a | 5.11E+06 | 3.15E+06 |
| MB/Silver | 82.39% | 8.54E+05 | 3.76E+05 |
| MB/Copper oxide | >99.99% (limit of | <2.00E+02 | 0 |
| | detection) | | |
| Crystal Violet/ZnO | >99.99% (limit of | <2.00E+02 | 0 |
| | detection) | | |
| Eosin-Y/ZnO | >99.99% (limit of | <2.00E+02 | 0 |
| | detection) | | |
| MB/ZnO | >99.99% (limit of | <2.00E+02 | 0 |
| | detection) | | |

TABLE 13
Testing against S. epidermidis in dark conditions.

| | | | Standard |
|---|---|---|---|
| | Mean % Log | Mean | deviation |
| Description | reduction | CFU/mL | CFU/mL |
| Inoculum | n/a | 7.81E+06 | 4.08E+06 |
| Control | n/a | 9.83E+06 | 3.92E+06 |
| MB/Silver | 82.88% | 1.77E+06 | 9.97E+05 |
| MB/Copper oxide | >99.99% (limit | <2.00E+02 | 0 |
| | of detection) | | |
| Crystal Violet/ZnO | 99.92% | 1.41E+05 | 1.99E+05 |
| Eosin-Y/ZnO | 96.03% | 3.76E+05 | 4.95E+04 |
| MB/ZnO | 92.53% | 7.65E+05 | 4.17E+05 |

### Example 5: Extrusion of Methylene Blue Antimicrobial Compositions

### Materials and Methods

In another example, extruded antimicrobial materials are produced with zinc oxide, methylene blue, and PLA antimicrobial mix. All PLA was dried in an oven at 60° C. for four hours and stored in a desiccator over calcium chloride ready for use. Methylene blue was sieved through an 80 mesh prior to use. In this experiment, samples were prepared using different ratios of three components, methylene blue (MB), zinc oxide (ZnO), and PLA, as shown in Table 14.

In this particular experiment, the nanoparticle and photosensitizer are dry-mixed with the PLA pellets in the hopper of a single-screw extruder. The MB and ZnO were mixed until uniform. This dry mix was added to PLA pellets. The final total mass of sample was 10 g. The extruder was set to 170° C. Once at temperature, the mixture of MB, ZnO, and PLA was placed into the hopper. The crank was turned clockwise at around 1 revolution per second. The molten extruded PLA mixture was retrieved at the end of the die/exit/spout. After roughly two pennies worth of PLA was extruded, a cut was made using a chisel/scissors. The newly extruded extruded polymer was collected, and another cut was made so that a pea sized amount was collected. The collected amount of extruded PLA was covered in foil and squeezed until flat. The samples were allowed to cool.

Cooled samples were washed once with phosphate buffered saline and twice with deionized water. Each wash was 5 minutes in duration at a temperature of 45° C. All materials were tested against E. coli (ATCC 8739) using 5 μL test volume for the spot plates and 25 μL volume of inoculum onto the surface of each sample. Each sample was tested in triplicate for activity under both ambient light and dark conditions according to the procedure in Example 1.

TABLE 14

| Sample | PLA | Zinc | Methylene |
|---|---|---|---|
| (% MB w/w) | (g) | Oxide (mg) | Blue (mg) |
| 0 | 10 | 0 | 0 |
| 0.05 | 9.975 | 200 | 5 |
| 0.1 | 9.97 | 200 | 10 |
| 0.25 | 9.775 | 200 | 25 |
| 0.5 | 9.75 | 200 | 50 |
| 1 | 9.7 | 200 | 100 |
| 2 | 9.6 | 200 | 200 |

### Results

Table 15 shows antimicrobial testing results for extruded polymers over increasing concentrations of MB from 0.05% to 2%. Table 15 describes the formulations of the extruded PLA polymers and their % log reduction of E. coli (ATCC 8739) relative to a blank control. FIG. 3 shows that when a polymer is extruded with antimicrobial compositions, it retains the deep blue coloration of methylene blue, confirming dispersion within the extruded polymer. FIG. 4 shows that when a polymer is extruded with antimicrobial compositions, antimicrobial activity was seen against E. coli ATCC 8739.

TABLE 15

| Sample (% MB W/W) | CFU/mL | % Reduction |
|---|---|---|
| Inoculum | 3.59E+08 | 0% |
| 0 | 9.06E+06 | 0% |
| 0.05 | 4.94E+06 | 70.32% |
| 0.1 | 4.00E+06 | 72.90% |
| 0.25 | 1.44E+06 | 91.23% |
| 0.5 | 1.81E+06 | 72.26% |
| 1 | 2.69E+06 | 79.35% |
| 2 | 5.25E+04 | 99.46% |

### Example 6: Extruded PLA Polymer with Mixtures of Photosensitizers

### Materials and Methods

Preparation of extruded polymer was performed as described previously in Example 5 using different mixtures of Rose Bengal (RB), and RB+MB mixtures along with 20 nm ZnO nanoparticles. Mixtures were 2% w/w ZnO with either 2% w/w RB, or 1% w/w RB+1% w/w MB. Extruded materials were then tested for antimicrobial activity against E. coli (ATCC 8739). All materials were tested in light conditions, and materials containing both MB and RB were tested in dark conditions as well according to the procedure in Example 1.

### Results

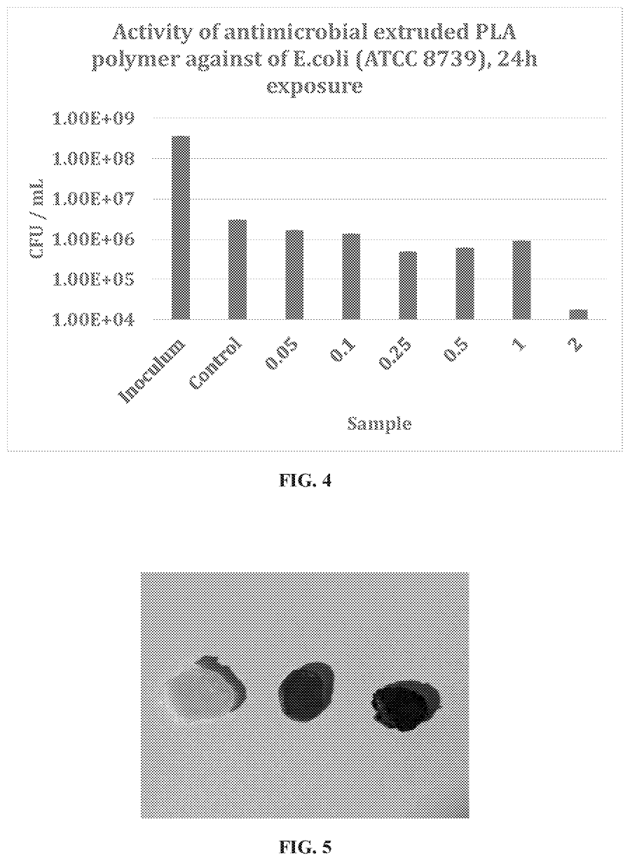

FIG. 5 shows extruded materials containing no antimicrobial mixture (left), a mixture of 2% w/w RB and 2% w/w ZnO (middle), and a mixture of 1% w/w RB, 1% w/w MB, and 2% w/w ZnO. The color change between samples indicates the RB+ZnO mixture and the RB+MB+ZnO mixtures are successfully embedded during the extrusion process even when high temperatures are used. Table 16 contains data indicating antimicrobial activity of extruded antimicrobial materials containing RB and ZnO or RB, MB, and ZnO mixtures. These data indicate the antimicrobial mixtures perform well after extrusion into PLA.

TABLE 16

| Sample (% w/w) | % reduction |
|---|---|
| Inoculum | 0% |
| Control | 0% |
| Light: 2% RB + 2% ZnO | >99% |
| Light: 1% RB + 1% MB + 2% ZnO | >99.99% |
| Dark: 1% RB + 1% MB + 2% ZnO | >90% |

### Example 7: Extruded PLA Polymer with Different Sizes and Amounts of ZnO Nanoparticles

### Materials and Methods

Preparation of extruded polymer was performed as described previously in Example 5, with varying sizes of ZnO nanoparticles. For this particular experiment, ZnO was 2% w/w and MB was 2% w/w. All materials were tested against E. coli (ATCC 8739). Each sample was tested in triplicate for activity after 24 h exposure under both ambient light and dark conditions according to the procedure in Example 1.

Further, the amount of ZnO used was analyzed, using 2% MB and varying amounts of ZnO (2%, 4%, and 8%). Samples were prepared as in Example 2 and tested against E. coli as in Example 1. The results of this experiment are presented below.

### Results

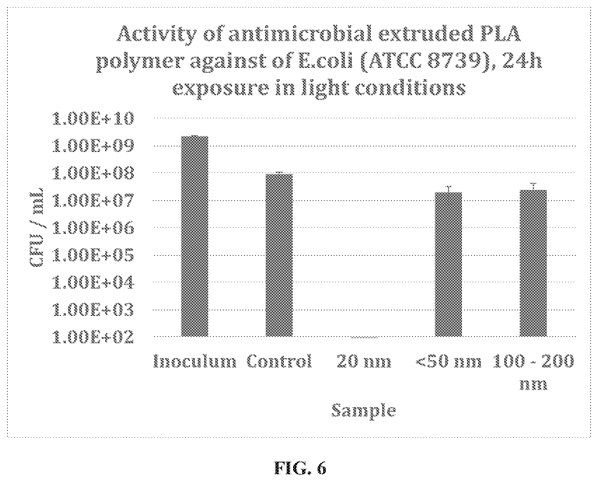

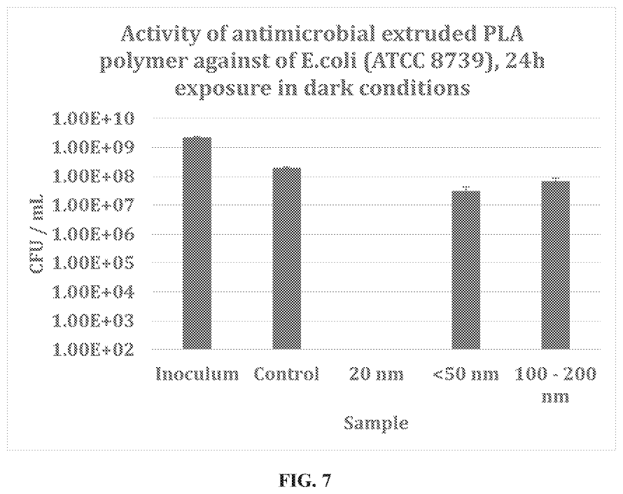

FIG. 6 and Table 17 show the activity of antimicrobial extruded PLA polymer with varying sizes of ZnO nanoparticles after 24 hours of exposure to E. coli (ATCC 8739) in light conditions. The sample with the 20 nm ZnO nanoparticles displayed the greatest antimicrobial activity with >99.999% reduction. FIG. 7 and Table 18 show the activity of the antimicrobial PLA polymer with varying ZnO nanoparticle sizes under dark conditions. Similar to in light conditions, the 20 nm ZnO nanoparticle shows >99.999% reduction, indicating that the antimicrobial polymer compositions are effective in both light and dark conditions.

TABLE 17

| Sample (ZnO size) | CFU/mL | % reduction |
|---|---|---|
| Inoculum | 2.19E+09 | |
| Control | 9.03E+07 | |
| 20 nm (light) | 1.00E+02 | >99.999% |
| | detection level | |
| <50 nm (light) | 1.98E+07 | 79.70% |
| 100-200 nm (light) | 2.37E+07 | 69.39% |

TABLE 18

| Sample (ZnO size) | CFU/mL | % reduction |
|---|---|---|
| Inoculum | 2.19E+09 | |
| Control | 1.93E+08 | |
| 20 nm (light) | 0.00E+00 | >99.999% |
| <50 nm (light | 3.02E+07 | 66.66% |
| 100-200 nm (light) | 6.71E+07 | 67.99% |

As described above, varying amounts of ZnO nanoparticles were used to generate PLA samples, which were tested against E. coli. The results of this experiment are as follows. With 2% ZnO and 2% MB in light conditions, >99.999% reduction was observed. Under dark conditions, with 2% MB and 2% ZnO, 89.6% reduction was observed. Under dark conditions with 2% MB and 4% ZnO, 98.8% reduction was observed. Under dark conditions with 2% MB and 8% ZnO, >99% reduction was observed.

### Example 8: Alternative Plastics

Polybutylene succinate and ethylene-vinyl acetate (EVA) were also tested with the antimicrobial compositions.

Polybutylene succinate (9 g) was added to an aluminum crucible. The crucible was heated to 140° C. and the molten polybutylene succinate was mixed at 30 rpm. Methylene blue (MP), 2% by weight, and zinc oxide (ZnO), 8% by weight, were added to the crucible. The resulting material was mixed manually for 20 minutes. Once mixing was completed, the resulting polybutylene succinate mixture was cut to shape and allowed to cool in air.

Ethylene-vinyl acetate (EVA) (9 g) was added to an aluminum crucible. The crucible was heated to 110° C. and the molten EVA was mixed at 30 rpm. Methylene blue (MP), 2% by weight, and zinc oxide (ZnO), 8% by weight, were added to the crucible. The resulting material was mixed for 20 minutes. Once mixing was completed, the resulting EVA mixture was cut to shape and allowed to cool in air.

These materials were tested as described in Experiment 1 against E. coli.

The results of these experiments are given in Tables 19-22. Similar performance was observed with the MB/ZnO antimicrobial composition mixture across PCL, EVA, and polybutylene succinate, suggesting that this application is viable for a broad spectrum of plastics.

TABLE 19
Testing polybutylene succinate in light
conditions against E. coli.

| | | | Standard |
|---|---|---|---|
| | Mean % Log | Mean | deviation |
| Description | reduction | CFU/mL | CFU/mL |
| Inoculum | n/a | 3.91E+08 | 1.55E+07 |
| Control | n/a | 8.01E+07 | 3.15E+06 |
| Polybutylene | >99.99% (limit | <2.00E+02 | 0 |
| succinate | of detection) | | |

TABLE 20
Testing polybutylene succinate in dark
conditions against E. coli.

| | | | Standard |
|---|---|---|---|
| | Mean % Log | Mean | deviation |
| Description | reduction | CFU/mL | CFU/mL |
| Inoculum | n/a | 4.85E+08 | 7.50E+06 |
| Control | n/a | 9.85E+08 | 5.80E+08 |
| Polybutylene | 79.35% | 2.25E+08 | 1.81E+08 |
| succinate | | | |

TABLE 21
Testing EVA in light conditions against E. coli.

| | Mean % Log | Mean | Standard deviation |
|---|---|---|---|
| Description | reduction | CFU/mL | CFU/mL |
| Inoculum | n/a | 3.91E+08 | 1.55E+07 |
| Control | n/a | 1.03E+08 | 9.35E+07 |
| EVA | >99.99% (limit | <2.00E+02 | 0 |
| | of detection) | | |

TABLE 22
Testing EVA in dark conditions against E. coli.

| | Mean % Log | Mean | Standard deviation |
|---|---|---|---|
| Description | reduction | CFU/mL | CFU/mL |
| Inoculum | n/a | 3.91E+08 | 1.55E+07 |
| Control | n/a | 2.28E+08 | 1.03E+07 |
| EVA | 76.34 | 5.45E+07 | 1.05E+07 |

### Example 9: Extruded PLA with Optimization for Broad Spectrum Kill

### Materials and Methods

Preparation of extruded polymer was performed as described previously in Example 2, with varying amounts of methylene blue, crystal violet, and zinc oxide, with the amounts indicated in the following tables. Each sample was tested in duplicate for activity after 24 h exposure under both ambient light and dark conditions according to the procedure in Example 1. The results of this experiment are presented below.

### Results

Tables 23-24 show the activity of antimicrobial extruded PLA polymer with indicated ingredients after 24 hours of exposure to E. coli (ATCC 8739) in light (Table 23) and dark (Table 24) conditions. Tables 25-26 show the activity of antimicrobial extruded PLA polymer with indicated ingredients after 24 hours of exposure to C. albicans in light (Table 25) and dark (Table 26) conditions.

TABLE 23

| | E. coli (light) | |
|---|---|---|
| Sample | CFU/mL | % reduction |
| Inoculum | 1.10E+09 | |
| Control | 9.83E+07 | |
| MB (1%) + CV (1%) + | 2.00E+02 | 99.99977.% |
| ZnO (8%) | | |
| CV (2%) + ZnO (8%) | 2.00E+02 | 99.99977.% |
| MB (2%) + ZnO (8%) | 2.00E+02 | 99.99977.% |

TABLE 24

| | E. coli (dark) | |
|---|---|---|
| Sample | CFU/mL | % reduction |
| Inoculum | 1.10E+09 | |
| Control | 4.74E+08 | |
| MB (1%) + CV (1%) + | 1.59E+06 | 99.995% |
| ZnO (8%) | | |
| CV (2%) + ZnO (8%) | 1.29E+07 | 98.142% |
| MB (2%) + ZnO (8%) | 7.14E+07 | 88.624% |

TABLE 25

| | C. albicans (light) | |
|---|---|---|
| | | % |
| Sample | CFU/mL | reduction |
| Inoculum | 1.10E+10 | |
| Control | 1.71E+08 | |
| MB (1%) + CV (1%) + | 9.04E+06 | 94.994% |
| ZnO (8%) | | |
| CV (2%) + ZnO (8%) | 8.81E+04 | 99.996% |
| MB (2%) + ZnO (8%) | 4.79E+07 | 82.481% |

TABLE 26

| | C. albicans (dark) | |
|---|---|---|
| | | % |
| Sample | CFU/mL | reduction |
| Inoculum | 1.10E+10 | |
| Control | 1.05E+08 | |
| MB (1%) + CV (1%) + | 2.01E+07 | 80.857% |
| ZnO (8%) | | |
| CV (2%) + ZnO (8%) | 1.44E+07 | 86.244% |
| MB (2%) + ZnO (8%) | 7.42E+07 | 29.124% |

### Example 10: Wound Drain

### Materials and Methods

In another example, extruded filaments were produced with zinc oxide and methylene blue antimicrobial mix. PLA pellets, with a melting point of 190° C., were dry mixed with methylene blue (MB) and zinc oxide (ZnO) nanopowder. In this particular experiment, the nanoparticle and photosensitizer were dry-mixed with the PLA pellets in the hopper of a single-screw extruder. Extrusion was conducted at 170° C. and was manually cranked to extrude the molten polymer mix into molds. The polymer mixture is then allowed to cool in the molds.

### Results

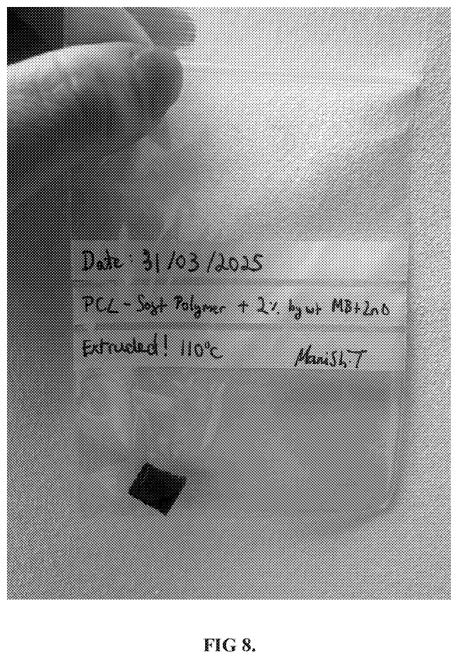

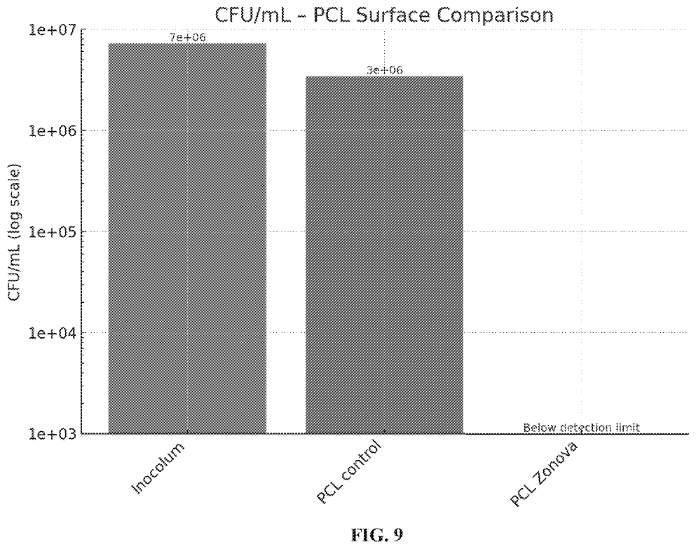

FIG. 8 shows that when a polymer is extruded with antimicrobial compositions, it retains the deep blue coloration of methylene blue, confirming dispersion within the extruded polymer. FIG. 9 shows that when a polymer is extruded with antimicrobial compositions, antimicrobial activity was seen against E. coli ATCC 25922 within 12 hours.

### Example 11: Mechanical Testing: Extruded Materials

Mechanical evaluation was performed on two PCL-based materials, including one sample MB/ZnO prepared as described in Example X and one control prepared in the same manner but without the addition of MB/ZnO. The tensile properties and surface hardness were determined to assess the impact of sample processing or formulation against the established control.

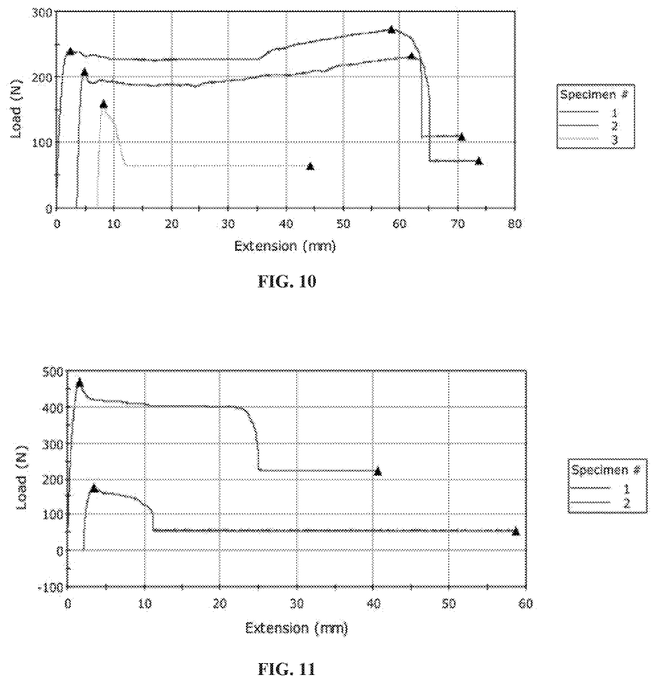

Tensile testing was conducted using an Instron 5569 equipped with a 50 kN load cell and a crosshead speed maintained at 10 mm/minute. Samples were conditioned for a minimum of 24 hours at an ambient temperature of approximately 24° C. prior to testing. Tensile testing data for the PCL control is shown in FIG. 10. Tensile testing data for the PCL sample is shown in FIG. 11.

Hardness testing measurements were taken using a Sauter Shore durometer (Shore D scale for hard plastics).

Mechanical tensile stress performance was maintained within experimental error.

### Example 12: Synthesis of Silicone Molding Materials

Pour silicone testing provides proof of concept that the technology described herein remains effective when embedded through silicone liquid injection molding manufacturing processes, validating that active additives survive without losing potency.

### Materials and Methods

A mixture of precipitated silica (6.2 g), silicone dioxide (3.7 g), and tetrapropyl orthosilicate (0.1 g) was placed in a suitable container. To this was added Rose Bengal lactone (RBL) (192 mg) and 50 nm zinc oxide (768 mg). This mixture was then stirred until the RBL and ZnO were fully dispersed within the matrix. Next, a mixture of precipitated silica (6.1 g), silicone dioxide (3.7 g), and Me hydrogen (0.2 g) were added, and the resulting viscous liquid was stirred until fully homogenous. The final material was allowed to cure overnight.

### Results

Various compositions of silicone were tested for antibacterial properties, as outlined in the following Examples. In these Examples, the best-performing compositions were determined. For Gram positive bacteria (Staphylococcus epidermidis), the best composition was found to be RBL with zinc oxide. For Gram negative bacteria (Escherichia coli), the best composition was found to be RBL with zinc oxide and crystal violet with zinc oxide. For fungi (Candida albicans), the best compositions were found to use RBL as the photosensitizer and iron (II,III) oxide as the metallic nanoparticle.

Further, when silicone was tested with ZnO on its own (no RBL), no killing in light or dark conditions was observed, indicating that the Rose Bengal lactone was necessary.

### Example 13: Variation of Photosensitizer in Silicone Materials

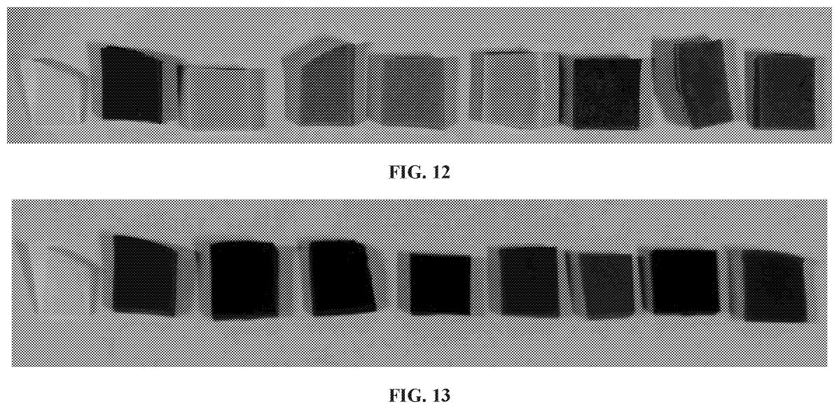

Using the process as described in Example 8, the photosensitizer (RBL) was exchanged with the following compounds: 1) Tris(2,2′-bipyridine)ruthenium(II) hexafluorophosphate; 2) Quercetin hydrate; 3) Curcumin; 4) Zn(II) Phthalocyanine; 5) Crystal Violet; 6) Azure-A; 9) Eosin-Y disodium salt. The products of this process are represented in FIG. 12, showing bone cement with MB replacements. From left to right, FIG. 12 shows: silicone (without any methylene blue, ZnO, or replacements), RBL, Tris(2,2′-bipyridine)ruthenium(II) hexafluorophosphate, Quercetin hydrate, Curcumin, Zn(II) Phthalocyanine, Protoporphyrin, Crystal Violet, Azure-A, and Eosin Y disodium salt.

Antibacterial testing was conducted with all materials described above. Antibacterial testing was carried out against Escherichia coli (ATCC 8739), Candida albicans (ATCC 10231), and Staphylococcus epidermidis (ATCC 12228). Each test was carried out in biological duplicate.

Samples tested for performance in the light were left on a table top within a room lit by LED lights at Ca. 250-350 Lux. Samples tested in the dark were left in the absence of light by placing them into a closed cabinet.

All test samples were left at room temperature overnight. Once complete, the solution was resuspended and serial dilutions were spot-plated to determine bacterial numbers.

### Results

Each of Escherichia coli, Candida albicans, and Staphylococcus epidermidis were tested against the compositions described above in both light and dark conditions. Tables 27 and 28 describe testing against E. coli. Table 29 describes testing against S. epidermidis in dark conditions.

TABLE 27
Testing in light conditions against E. coli.

| | | | Standard |
|---|---|---|---|
| | Mean % Log | Mean | deviation |
| Description | reduction | CFU/mL | CFU/mL |
| Inoculum | n/a | 1.09E+09 | 1.80E+08 |
| Control | n/a | 1.61E+08 | 8.13E+07 |
| Tris(2,2′- | 95.06% | 2.63E+07 | 3.38E+07 |
| bipyridine) | | | |
| ruthenium(II) | | | |
| hexaflurophosphate | | | |
| Quercetin hydrate | 83.70% | 4.42E+07 | 4.96E+07 |
| Curcumin | 98.53% | 3.01E+06 | 2.14E+06 |
| Zinc phthalocyanine | 97.50% | 5.64E+06 | 6.32E+06 |
| Crystal violet | Below limit of | <2.00E+02 | 0 |
| | detection | | |
| | (>99.99%) | | |
| Azure A chloride | Below limit of | <2.00E+02 | 0 |
| | detection | | |
| | (>99.99%) | | |
| Eosin Y disodium salt | 99.97% | 2.19E+05 | 2.91E+05 |
| Rose Bengal lactone | Below limit of | <2.00E+02 | 0 |
| | detection | | |
| | (>99.99%) | | |

TABLE 28
Testing in dark conditions against E. coli.

| | Mean % | | Standard |
|---|---|---|---|
| | Log | Mean | deviation |
| Description | reduction | CFU/mL | CFU/mL |
| Inoculum | n/a | 1.09E+09 | 1.80E+08 |
| Control | n/a | 1.81E+08 | 1.25E+08 |
| Tris(2,2′- | 55.33% | 6.65E+07 | 1.22E+07 |
| bipyridine)ruthenium | | | |
| (II)hexaflurophosphate | | | |
| Quercetin hydrate | 17.14% | 1.29E+08 | 5.41E+07 |
| Curcumin | 91.30% | 1.28E+07 | 4.54E+05 |
| Zinc phthalocyanine | 97.11% | 4.94E+06 | 3.24E+06 |
| Crystal violet | 91.50% | 1.25E+07 | 8.41E+05 |
| Azure A chloride | 95.35% | 6.86E+06 | 4.54E+05 |
| Eosin Y disodium salt | 90.29% | 1.44E+07 | 1.93E+06 |
| Rose Bengal lactone | 94.70% | 1.69E+07 | 2.35E+07 |

TABLE 29
Testing in dark conditions against S. epidermidis.

| | | | Standard |
|---|---|---|---|
| | Mean % Log | Mean | deviation |
| Description | reduction | CFU/mL | CFU/mL |
| Inoculum | n/a | 7.81E+06 | 4.08E+06 |
| Control | n/a | 2.21E+06 | 1.20E+05 |
| Crystal Violet/ZnO | 99.87% | 2.13E+04 | 2.98E+04 |
| Eosin-Y/ZnO | 87.00% | 3.73E+05 | 3.37E+05 |
| RBL/ZnO | >99.99% (Limit of | <2.00E+02 | 0.00E+00 |
| | detection) | | |

### Example 14: Variation of Metallic Nanoparticles in Silicone Materials

Using the process as described in Example 9, the metallic nanoparticle (zinc oxide) was exchanged with the following at the same percentage weight: 1) silver nanoparticles (<100 nm); 2) copper nanoparticles (40-60 nm); 3) cupric oxide nanoparticles (<50 nm); 4) magnesium oxide nanoparticles (50 nm); 5) tungsten (VI) oxide nanoparticles (<100 nm); 6) iron (II,III) oxide nanoparticles (50-100 nm); 7) graphene oxide powder (average number of layers 15-20); and 8) titanium dioxide nanoparticles (<100 nm). The products of this process are represented in FIG. 13, showing silicone with ZnO replacements. From left to right, FIG. 13 shows: silicone (without any RBL, ZnO, or replacements), ZnO, silver nanoparticles, copper nanoparticles, cupric oxide nanoparticles, magnesium oxide nanoparticles, tungsten (VI) oxide nanoparticles, iron (II,III) oxide nanoparticles, and titanium dioxide nanoparticles.

Antibacterial testing was conducted with all materials described above. Antibacterial testing was carried out against Escherichia coli (ATCC 8739), Candida albicans (ATCC 10231), and Staphylococcus epidermidis (ATCC 12228). Each test was carried out in biological duplicate.

Samples tested for performance in the light were left on a table top within a room lit by LED lights at Ca. 250-350 Lux. Samples tested in the dark were left in the absence of light by placing them into a closed cabinet.

All test samples were left at room temperature overnight. Once complete, the solution was resuspended and serial dilutions were spot-plated to determine bacterial numbers.

### Results

Each of Escherichia coli, Candida albicans, and Staphylococcus epidermidis were tested against the compositions described above in both light and dark conditions. Tables 30 and 31 describe testing against E. coli. Table 32 describes testing against S. epidermidis in dark conditions.

TABLE 30
Testing in light conditions against E. coli.

| | | | Standard |
|---|---|---|---|
| | Mean % Log | Mean | deviation |
| Description | reduction | CFU/mL | CFU/mL |
| Inoculum | n/a | 4.24E+08 | 2.40E+07 |
| Control | n/a | 2.28E+08 | 4.61E+07 |
| Silver (<100 nm) | >99.99% (limit of | <2.00E+02 | 0 |
| | detection) | | |
| Copper (40-60 nm) | >99.99% (limit of | <2.00E+02 | 0 |
| | detection) | | |
| Copper (II) Oxide | >99.99% (limit of | <2.00E+02 | 0 |
| (<50 nm) | detection) | | |
| Magnesium Oxide | >99.99% (limit of | <2.00E+02 | 0 |
| (<50 nm) | detection) | | |
| Tungsten (VI) Oxide | >99.99% (limit of | <2.00E+02 | 0 |
| (<100 nm) | detection) | | |
| Iron (II, III) Oxide | >99.99% (limit of | <2.00E+02 | 0 |
| (50-100 nm) | detection) | | |
| Titanium Dioxide | >99.99% (limit of | <2.00E+02 | 0 |
| | detection) | | |
| Zinc Oxide (100- | >99.99% (limit of | <2.00E+02 | 0 |
| 200 nm) | detection) | | |

TABLE 31
Testing in dark conditions against E. coli.

| | Mean % | | Standard |
|---|---|---|---|
| | Log | Mean | deviation |
| Description | reduction | CFU/mL | CFU/mL |
| Inoculum | n/a | 4.24E+08 | 2.40E+07 |
| Control | n/a | 3.74E+08 | 7.40E+07 |
| Silver (<100 nm) | 82.78% | 7.05E+07 | 1.46E+04 |
| Copper (40-60 nm) | 99.97% | 9.17E+04 | 8.32E+03 |
| Copper (II) Oxide | >99.99% | 9.40E+03 | 4.07E+03 |
| (<50 nm) | | | |
| Magnesium Oxide | 86.47% | 5.10E+07 | 1.18E+07 |
| (<50 nm) | | | |
| Tungsten (VI) Oxide | 51.73% | 2.09E+08 | 1.17E+08 |
| (<100 nm) | | | |
| Iron (II, III) Oxide | 80.38% | 1.53E+07 | 2.37E+06 |
| (50-100 nm) | | | |
| Titanium Dioxide | 84.15% | 1.23E+07 | 1.93E+06 |
| Zinc Oxide (100- | 97.19% | 3.45E+07 | 2.96E+07 |
| 200 nm) | | | |

TABLE 32
Testing in dark conditions against S. epidermidis.

| | | | Standard |
|---|---|---|---|
| | Mean % Log | Mean | deviation |
| Description | reduction | CFU/mL | CFU/mL |
| Inoculum | n/a | 7.81E+06 | 4.08E+06 |
| Control | n/a | 2.21E+06 | 1.20E+05 |
| MB/Silver | >99.99% (Limit of | <2.00E+02 | 0.00E+00 |
| | detection) | | |
| MB/Copper oxide | >99.99% (Limit of | <2.00E+02 | 0.00E+00 |
| | detection) | | |
| Crystal Violet/ZnO | 99.87% | 2.13E+04 | 2.98E+04 |
| Eosin-Y/ZnO | 87.00% | 3.73E+05 | 3.37E+05 |
| RBL/ZnO | >99.99% (Limit of | <2.00E+02 | 0.00E+00 |
| | detection) | | |

This process can also be completed with commercial silicone mold making kits. Specifically, Let's Resin Silicone Mole Making Kit was used. This kit provides an “A” and “B” solution which, when mixed, leads to solid silicone. Preparation of antimicrobial materials are produced with zinc oxide, methylene blue, and silicone antimicrobial mix. MB (192 mg) and ZnO (768 mg) were ground with the “A” solution containing vinyl silicone oil (CAS 112926-00-8) 62% w/w, silicon dioxide (CAS 7631-86-9) 37% w/w, and tetrapropyl orthosilicate (CAS 682-01-9) 1% w/w to generate a slurry. This was done for 10, 15, and 20 minutes to observe the effect grinding time had on the dispersion of MB in the silicone. The slurry was then mixed with the “B” solution containing vinyl silicone oil (CAS 112926-00-8) 61% w/w, silicon dioxide (CAS 7631-86-9) 37% w/w, and poly(methylhydrosiloxane) (CAS 63148-57-2) 2% w/w. This generates solid silicone material. This process was also repeated with Rose Bengalbengal, again using 192 mg.

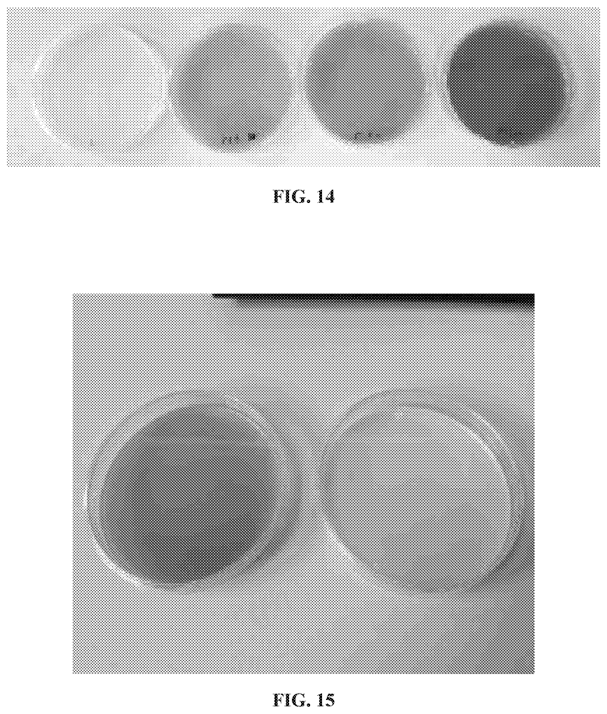

FIG. 14 shows silicone samples which were subjected to different grinding times. The effect of grinding times can be seen by a change in the intensity of blue, where the silicone only (left) shows no blue, the samples which were subjected to grinding for 10 and 15 minutes (middle) show a light blue, and the sample ground for 20 minutes (right) shows a darker blue. The change in color is due to MB becoming more dispersed throughout the silicone with the longer grinding times. FIG. 15 shows the prepared Rose Bengal silicone (left) compared to silicone only (right). The color change to pink indicates dispersion of Rose Bengal throughout the silicone.

### Example 15: Mechanical Testing: Silicone Materials

Mechanical evaluation was performed on two silicone-based materials, including one sample RBL/ZnO prepared as described in Example 9 and one control prepared in the same manner but without the addition of RBL/ZnO. The tensile properties and surface hardness were determined to assess the impact of sample processing or formulation against the established control.

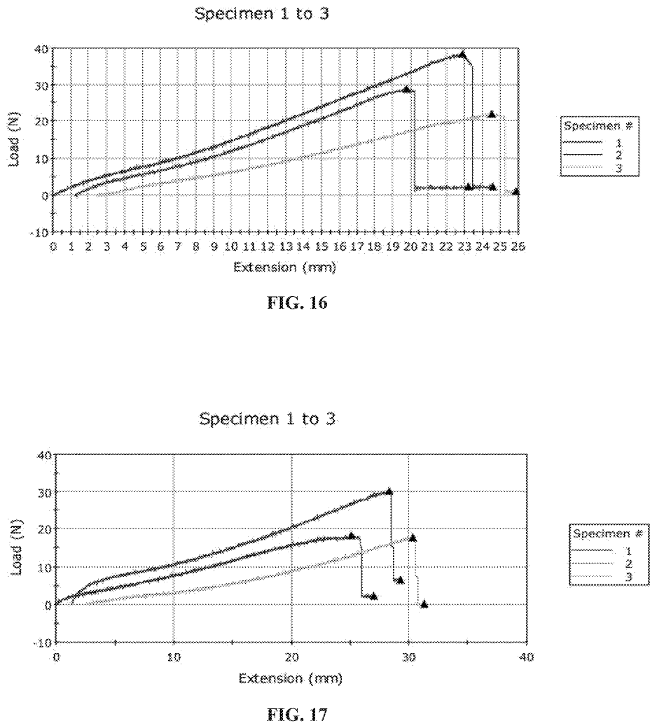

Tensile testing was conducted using an Instron 5569 equipped with a 50 kN load cell and a crosshead speed maintained at 10 mm/minute. Samples were conditioned for a minimum of 24 hours at an ambient temperature of approximately 24° C. prior to testing. Tensile testing data for the silicone control is shown in FIG. 16. Tensile testing data for the silicone sample is shown in FIG. 17.

Mechanical tensile stress performance was maintained within experimental error.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples are provided for clarity of understanding only. No unnecessary limitations are to be understood therefrom. The invention is not limited to the exact details shown and described, for variations obvious to one skilled in the art will be included within the invention defined by the claims.

What is claimed is:

1. An antimicrobial polymer composition comprising:
a) a plurality of metallic nanoparticles;
b) one or more photosensitizers; and
c) a polymer;
wherein the metallic nanoparticles and one or more photosensitizers are mixed to form a metallic nanoparticle-photosensitizer mixture;
wherein the metallic nanoparticle-photosensitizer mixture is combined with the polymer under high temperature or pressure conditions to form a medical device; and
wherein the medical device exhibits antimicrobial activity under both light and dark conditions without requiring a light-activation source.

2. The antimicrobial polymer composition of claim 1, wherein the plurality of metallic nanoparticles are selected from a group consisting of zinc oxide, zinc, gold, silver, copper, cupric oxide, magnesium oxide, iron (II,III) oxide, graphene oxide, titanium dioxide, tungsten (VI) oxide nanoparticles, alloys of two or more of these metals, or a combination thereof.

3. The antimicrobial polymer composition of claim 1, wherein neither the plurality of metallic nanoparticles nor the one or more photosensitizers leaches from the composition.

4. The antimicrobial polymer composition of claim 1, wherein the photosensitizer comprises about 0.05% to about 5% by weight of the total polymer.

5. The antimicrobial polymer composition of claim 1, wherein the metallic nanoparticles comprise about 1% to about 10% by weight of the total polymer.

6. The antimicrobial polymer composition of claim 1, wherein the plurality of nanoparticles comprises nanoparticles of less than 100 nm in diameter.

7. The antimicrobial bone cement composition of claim 1, wherein the one or more photosensitizers are selected from the group consisting of toluidine blue O, methylene blue, dimethylmethylene blue, zinc phthalocyanine, silicon phthalocyanine, aluminum phthalocyanine, Rose Bengal, porphyrin, haematoporphyrin, deuteroporphyrin, tin chlorin e6, poly-lysine derivatives of tin chlorin e6, m-tetrahydroxyphenyl chlorin, benzoporphyrin derivatives, tin etiopurpurin, bacteriochlorins, phenazines, acriflavine, proflavin, acridine orange, aminacrine, texaphyrins, cyanines including merocyanine 540, anthracyclins including adriamycin and epirubicin, pheophorbides, sapphyrins, fullerene, perylenequinonoid pigments including hypericin and hypocrellin, gilvocarcins, terthiophenes, benzophenanthridines, tris(2,2′-bipyridine)ruthenium(II) hexafluorophosphate, quercetin hydrate, curcumin, Zn(II) phthalocyanine, eosin-Y disodium salt, psoralens and riboflavin arianor steel blue, tryptan blue, crystal violet, azure blue cert, azure B chloride, azure 2, azure A chloride, azure B tetrafluoroborate, thionin, azure A eosinate, azure B eosinate, azure mix sicc, and azure II eosinate, or a combination thereof.

8. The antimicrobial polymer composition of claim 1, wherein the photosensitizer is methylene blue, Rose Bengal, tris(2,2′-bipyridine)ruthenium(II) hexafluorophosphate, quercetin hydrate, curcumin, Zn(II) Phthalocyanine, crystal Violet, azure-A, eosin-Y disodium salt, or a combination thereof.

9. The antimicrobial polymer composition of claim 1, wherein the metallic nanoparticle-photosensitizer mixture is a zinc oxide-methylene blue mixture and the mixture is dispersed within the composition.

10. The antimicrobial polymer composition of claim 1, wherein the metallic nanoparticle-photosensitizer mixture is a zinc oxide-Rose Bengal mixture.

11. The antimicrobial polymer composition of claim 1, wherein the high temperatures are temperatures of between 150° C. and 350° C.

12. The antimicrobial polymer composition of claim 1, wherein the composition generates reactive oxygen species.

13. The antimicrobial polymer composition of claim 1, wherein the polymer is selected from the group consisting of polyvinyl chloride, polylactic acid, polybutylene succinate, ethylene-vinyl acetate, polyethylene, ultra-high-molecular-weight polyethylene, polypropylene, polystyrene, polyethylene terephthalate, polybutylene terephthalate, nylon, polymethyl methacrylate, polycarbonate, polyoxymethylene, acetal, thermoplastic polyurethane, silicone, high consistency rubber, styrene-ethylene-butylene-styrene, polyisoprene, polytetrafluoroethylene, fluorinated ethylene propylene, polyvinylidene fluoride, polybutylenesuccinate homopolymer, polybutylene adipate homopolymer, polybutyleneadipate-succinate copolymer, polyethylenesuccinate-adipate copolymer, polyethylene glycol succinate and polyethylene adipate homopolymer, poly(lactide), poly(glycolide), poly(lactide-co-glycolide), poly(L-lactide-co-trimethylene carbonate), poly(dioxanone), poly(butylene succinate), poly(butylene adipate), polyactic acid, poly(lactic-co-glycolic acid), polyglycolic acid, polycaprolactone, polydioxanone, polyanhydrides, polyorthoesters, polyether ketone, polysulfones, polyaryletherketones, polyethylene terephthalate based copolymers, liquid crystal copolymers, collagen, gelatin, chitosan, cellulose, cellulose acetate, carboxymethyl cellulose, hydroxypropyl methylcellulose, silk fibroin, alginate, and combinations thereof.

14. A method for manufacturing an antimicrobial polymer of claim 1 comprising the steps of:
a) providing a mixture of a plurality of metallic nanoparticles and one or more photosensitizers to produce a metallic nanoparticle-photosensitizer mixture;
b) combining a polymer with the metallic nanoparticle-photosensitizer mixture;
c) heating the combined metallic nanoparticle-photosensitizer mixture and polymer to produce a molten mixture; and
d) forming the molten mixture into a desired shape.

15. The method of claim 14, wherein the molten mixture is formed into a shape of a medical device by extrusion, injection molding, pouring, or a combination thereof.

16. The method of claim 14, wherein the combined metallic nanoparticle-photosensitizer mixture is heated to between 150° C. and 350° C.

17. The method of claim 14, wherein the combined metallic nanoparticle-photosensitizer mixture and polymer is heated within an extruder.

18. The method of claim 14, further comprising a step of transferring the molten mixture from an extruder barrel to a shaped cavity, wherein the shaped cavity forms the molten mixture into the desired shape.

19. The method of claim 14, wherein the desired shape is the shape of a medical device.
